(19) United States
(12) Patent Application Publication
(10) Pub. No.: US 2025/0197405 A1
(43) Pub. Date: Jun. 19, 2025

(54) HETEROAROMATIC COMPOUNDS AS PKMYT1 INHIBITORS AND USE THEREOF

(71) Applicant: INSILICO MEDICINE IP LIMITED, Hong Kong (CN)

(72) Inventors: Yazhou Wang, Pudong New Area, Shanghai (CN); Xiao Ding, Pudong New Area, Shanghai (CN); Min Zheng, Pudong New Area, Shanghai (CN); Feng Ren, Pudong New Area, Shanghai (CN)

(73) Assignee: INSILICO MEDICINE IP LIMITED, Hong Kong (CN)

(21) Appl. No.: 18/846,613

(22) PCT Filed: Mar. 15, 2023
(86) PCT No.: PCT/CN2023/081618

§ 371 Date: Sep. 12, 2024

(30) Foreign Application Priority Data
Mar. 15, 2022 (WO) PCT/CN2022/080868
Aug. 9, 2022 (WO) PCT/CN2022/111131

(51) Int. Cl. C07D 487/04; A61K 31/437; A61K 31/519; C07D 471/04

(52) U.S. Cl. CPC C07D 487/04; A61K 31/437; A61K 31/519; C07D 471/04

(57) ABSTRACT

Providing compounds of Formula (A) or pharmaceutically acceptable salts, solvate, stereoisomer, or isotopic variant thereof useful as PKMYT1 inhibitors, pharmaceutical compositions comprising the same, and use thereof in the manufacture of medicaments for treating PKMYT1-associated diseases or conditions such as advanced solid tumor, and etc.

### CROSS-REFERENCE TO RELATED APPLICATIONS

The present application claims priority to PCT Application No. PCT/CN2022/080868 filed on Mar. 15, 2022, entitled “HETEROAROMATIC COMPOUNDS AS PKMYT1 INHIBITORS AND USE THEREOF” and PCT Application No. PCT/CN2022/111131 filed on Aug. 9, 2022, entitled “HETEROAROMATIC COMPOUNDS AS PKMYT1 INHIBITORS AND USE THEREOF”, the disclosures of which are hereby incorporated by reference in its entirety.

### FIELD

The present disclosure relates to novel heteroaromatic compounds or pharmaceutically acceptable salts, stereoisomer, or isotopic variant thereof, which are useful as PKMYT1 inhibitors. The present disclosure further relates to pharmaceutical compositions comprising one or more of such compounds or pharmaceutically acceptable salts, stereoisomer, or isotopic variant thereof as an active ingredient, and use of such compounds or pharmaceutically acceptable salts, stereoisomer, or isotopic variant thereof in the treatment of PKMYT1-associated diseases or conditions, such as tumor, particularly, advanced solid tumor, etc.

### BACKGROUND

DNA is continuously subjected to both endogenous insults (e.g., stalled replication forks, reactive oxygen species) and exogenous insults (UV, ionizing radiation, chemical) that can lead to DNA damage. As a result, cells have established sophisticated mechanisms to counteract these deleterious events that would otherwise compromise genomic integrity and lead to genomic instability diseases such as tumor. To maintain genomic integrity, two checkpoints of cell cycle allow for efficient repair of DNA damages, one of which is the WEE kinase family. The WEE kinase family consists of three members: WEE1, PKMYT1, and the less important WEE1B. PKMYT1 functions as negative regulators of the cell cycle by inhibiting the CDK1-cyclin B complex. CDK1, a master regulator of the cell cycle, is essential for entry into mitosis, meaning that PKMYT1 might be expected to act as tumor suppressors by preventing CDK1 activation. Studies showed that loss of PKMYT1 interferes with the G2-M checkpoint, driving cells into mitosis prematurely, which results in the accumulation of genetic lesions from unrepaired DNA damage, ultimately leading to apoptosis or mitotic catastrophe. However, the concomitant inhibition of WEE1 and PKMYT1 leads to strong cytotoxic effects. Thus, PKMYT1 is a promising target for anti-cancer therapy. Moreover, PKMYT1 has also been implicated in many cancer types, including gastric cancer, non-small-cell lung cancer, hepatocellular carcinoma, glioblastoma, neuroblastoma, and colorectal cancer, etc., in which overexpression of PKMYT1 generally correlates with poor prognosis and disease progression. Therefore, selective targeting PKMYT1 with small molecules provides new opportunities for cancer therapy.

### SUMMARY

Disclosed herein are novel heteroaromatic compounds as PKMYT1 inhibitors. As a result, the compounds of the present disclosure are particularly useful in the modulation of PKMYT1 and thus in the treatment of PKMYT1-associated diseases and conditions.

In one aspect, the present disclosure is directed to a compound of Formula

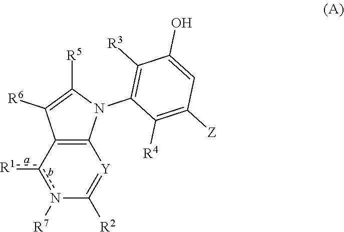

or a pharmaceutically acceptable salt, stereoisomer, or isotopic variant thereof, wherein [symbol] , R¹, R², R³, R⁴, R⁵, R⁶, R⁷, and Y are described herein.

In one aspect, the present disclosure is directed to a compound of Formula (I) or (II)

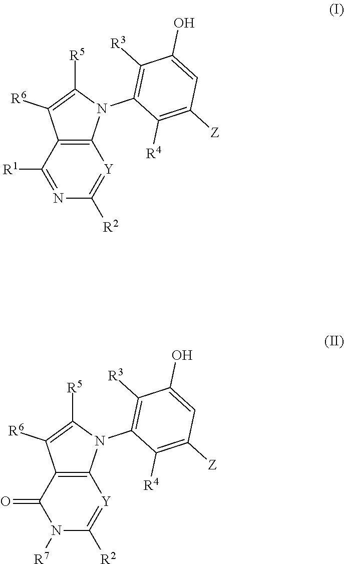

or a pharmaceutically acceptable salt, stereoisomer, or isotopic variant thereof, wherein R¹, R², R³, R⁴, R⁵, R⁶, R⁷, and Y are described herein.

In another aspect, the present disclosure is directed to a pharmaceutical composition for treating a PKMYT1-associated disease or condition, which comprises the compound of Formula (I) or (II) or a pharmaceutically acceptable salt, stereoisomer, or isotopic variant thereof as provided herein, and a pharmaceutically acceptable carrier or excipient.

In a further aspect, the present disclosure is directed to a method of treating a PKMYT1-associated disease or condition in a subject in need thereof, which comprises administering to the subject a therapeutically effective amount of the compound of Formula (I) or (II) or a pharmaceutically acceptable salt, stereoisomer, or isotopic variant thereof as provided herein.

In a further aspect, the present disclosure is directed to the compound of Formula (I) or (II) or a pharmaceutically acceptable salt, stereoisomer, or isotopic variant thereof as provided herein for use in the treatment of a PKMYT1-associated disease or condition.

In a further aspect, the present disclosure is directed to use of the compound of Formula (I) or (II) or a pharmaceutically acceptable salt, stereoisomer, or isotopic variant thereof as provided herein in the manufacture of a medicament for treating a PKMYT1-associated disease or condition.

In a further aspect, the present disclosure is directed to a kit for treating a PKMYT1-associated disease or condition, which comprises the compound of Formula (I) or (II) or a pharmaceutically acceptable salt, stereoisomer, or isotopic variant thereof as provided herein, a container, and optionally a package insert or label indicating treatment of said disease or condition.

### DETAILED DESCRIPTION

Reference will now be made in detail to certain embodiments, examples of which are illustrated in the accompanying detailed description. While enumerated embodiments will be described, it shall be understood that they are not intended to limit the present disclosure to those embodiments. On the contrary, the present disclosure is intended to cover all alternatives, modifications, and equivalents, which may be included within the scope of the present disclosure as defined by the claims. Those skilled in the art will recognize many methods and materials similar or equivalent to those described herein, which could be used in the practice of the present disclosure. The present disclosure is in no way limited to the methods and materials as described. In the event that one or more of the incorporated literatures and similar materials differs from or contradicts this disclosure, including but not limited to defined terms, term usage, described techniques, or the like, this disclosure controls.

It is appreciated that certain features of the present disclosure, which are, for clarity, described in the context of separate embodiments, can also be provided in combination in a single embodiment. Conversely, various features of the present disclosure, which are, for brevity, described in the context of a single embodiment, can also be provided separately or in any suitable sub-combination.

Accordingly, the followings are provided herein.

Item 1. a compound of Formula

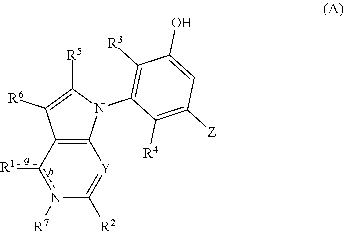

or a pharmaceutically acceptable salt, stereoisomer, or isotopic variant thereof,
wherein
   “ [symbol] ” represents a single or double bond, provided that when “ [symbol] ” is a single bond, “ [symbol] ” is a double bond, or when “ [symbol] ” is a double bond, “ [symbol] ” is a single bond;
   Y is N or CR⁸;
   Z is hydrogen, optionally substituted C₁₋₃ alkyl, optionally substituted C₃₋₅ cycloalkyl, or halogen;
   when “ [symbol] ” is a single bond, R¹ is independently hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted 3- to 10-membered heterocyclyl, optionally substituted 3- to 10-membered heterocyclyl C₁₋₆ alkyl, optionally substituted 6- to 10-membered aryl, optionally substituted 5- to 10-membered heteroaryl, optionally substituted 5- to 10-membered heteroaryl C₁₋₆ alkyl, halogen, cyano, —N(R^{A})₂, —OR^{A}, —SR^{A}, —C(O)N(R^{B})₂, —SO₂N(R^{B})₂, —SO₂R^{C}, oxo (═O), or -Q-R^{X}; R⁷ is absent;
   when “ [symbol] ” is a double bond, R¹ is independently O, S, or CH; R⁷ is hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted 3- to 10-membered heterocyclyl, optionally substituted 3- to 10-membered heterocyclyl C₁₋₆ alkyl, optionally substituted 6- to 10-membered aryl, optionally substituted 5- to 10-membered heteroaryl, optionally substituted 5- to 10-membered heteroaryl C₁₋₆ alkyl, halogen, cyano, —N(R^{A})₂, —OR^{A}, —C(O)N(R^{B})₂, —SO₂N(R^{B})₂, —SO₂R^{C}, or -Q-R^{X};
   each of R², and R⁸ is independently hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted 3- to 10-membered heterocyclyl, optionally substituted 3- to 10-membered heterocyclyl C₁₋₆ alkyl, optionally substituted 6- to 10-membered aryl, optionally substituted 5- to 10-membered heteroaryl, optionally substituted 5- to 10-membered heteroaryl C₁₋₆ alkyl, halogen, cyano, —N(R^{A})₂, —OR^{A}, —SR^{A}, —C(O)N(R^{B})₂, —SO₂N(R^{B})₂, —SO₂R^{C}, oxo (═O), or -Q-R^{X};
   each of R³ and R⁴ is independently optionally substituted C₁₋₆ alkyl, optionally substituted C₁₋₆ alkenyl, optionally substituted C₁₋₆ alkynyl, optionally substituted C₃₋₅ cycloalkyl, or halogen;
   R⁵ is H or —N(R^{A})₂;
   R⁶ is —C(O)NH(R^{B}), —C(O)R^{C}, or —SO₂R^{C};
   each R^{X} is independently hydroxyl, optionally substituted C₁₋₆ alkyl, optionally substituted 6- to 10-membered aryl, optionally substituted 3- to 10-membered heterocyclyl, optionally substituted 5- to 10-membered heteroaryl, —N(R^{A})₂, —C(O)N(R^{B})₂, —SO₂N(R^{B})₂, —SO₂R^{C}, or optionally substituted C₁₋₆alkoxy;
   each R^{A} is independently hydrogen, optionally substituted C₁₋₆ alkyl, optionally substituted 6- to 10-membered aryl C₁₋₆ alkyl, optionally substituted C₃₋₈ cycloalkyl, optionally substituted 6- to 10-membered aryl, optionally substituted 3- to 10-membered heterocyclyl, optionally substituted 5- to 10-membered heteroaryl, optionally substituted 5- to 10-membered heteroaryl C₁₋₆ alkyl, or —SO₂R^{C}; or two R^{A}, together with the atom to which they are attached, combine to form an optionally substituted 3- to 10-membered heterocyclyl;
   each R^{B} is independently hydrogen, optionally substituted C₁₋₆ alkyl, optionally substituted C₂₋₆ alkoxy C₁₋₆ alkyl, optionally substituted 6- to 10-membered aryl C₁₋₆ alkyl, optionally substituted C₃₋₈ cycloalkyl, optionally substituted 6- to 10-membered aryl, optionally substituted 3- to 10-membered heterocyclyl, or optionally substituted 5- to 10-membered heteroaryl; or two R^{B}, together with the atom to which they are attached, combine to form an optionally substituted 3- to 10-membered heterocyclyl;
   each R^{C} is independently optionally substituted C₁₋₆ alkyl, optionally substituted C₃₋₈ cycloalkyl, optionally substituted 6- to 10-membered aryl, optionally substituted 3- to 10-membered heterocyclyl, or optionally substituted 5- to 10-membered heteroaryl;
   Q is optionally substituted C₁₋₆ alkylene, optionally substituted C₂₋₆ alkenylene, optionally substituted C₂₋₆ alkynylene, optionally substituted C₃₋₈ cycloalkylene, optionally substituted C₃₋₈ cycloalkenylene, optionally substituted 6- to 10-membered arylene, optionally substituted 3- to 10-membered heterocyclylene, or optionally substituted 5- to 10-membered heteroarylene.

In certain embodiments, Z is hydrogen. In certain embodiments, Z is optionally substituted C₁₋₃ alkyl, optionally substituted C₃₋₅ cycloalkyl, or halogen.

In certain embodiments, each of R³ and R⁴ is independently optionally substituted C₁₋₆ alkyl or halogen. In certain embodiments, each of R³ and R⁴ is independently optionally substituted C₁₋₆ alkenyl, optionally substituted C₁₋₆ alkynyl, or optionally substituted C₃₋₅ cycloalkyl.

Item 2. The compound or a pharmaceutically acceptable salt, stereoisomer, or isotopic variant thereof according to Item 1, wherein the compound is of Formula (I) or (II)

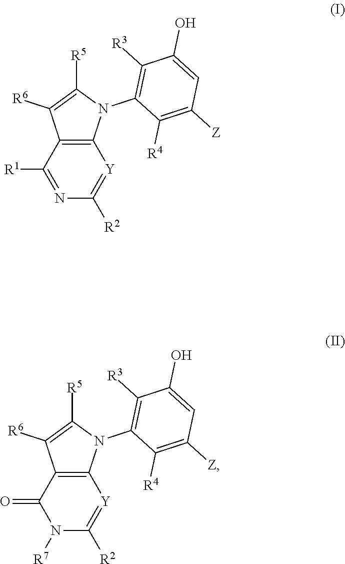

wherein
   Y is N or CR⁸;
   Z is hydrogen, optionally substituted C₁₋₃ alkyl, optionally substituted C₃₋₅ cycloalkyl, or halogen;
   each of R¹, R², and R³ is independently hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted 3- to 10-membered heterocyclyl, optionally substituted 3- to 10-membered heterocyclyl C₁₋₆ alkyl, optionally substituted 6- to 10-membered aryl, optionally substituted 5- to 10-membered heteroaryl, optionally substituted 5- to 10-membered heteroaryl C₁₋₆ alkyl, halogen, cyano, —N(R^{A})₂, —OR^{A}, —SR^{A}, —C(O)N(R^{B})₂, —SO₂N(R^{B})₂, —SO₂R^{C}, or -Q-R^{X};
   each of R³ and R⁴ is independently optionally substituted C₁₋₆ alkyl, optionally substituted C₁₋₆ alkenyl, optionally substituted C₁₋₆ alkynyl, optionally substituted C₃₋₅ cycloalkyl, or halogen;
   R⁵ is H or —N(R^{A})₂;
   R⁶ is —C(O)NH(R^{B}), —C(O)R^{C}, or —SO₂R^{C};
   R⁷ is hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted 3- to 10-membered heterocyclyl, optionally substituted 3- to 10-membered heterocyclyl C₁₋₆ alkyl, optionally substituted 6- to 10-membered aryl, optionally substituted 5- to 10-membered heteroaryl, optionally substituted 5- to 10-membered heteroaryl C₁₋₆ alkyl, halogen, cyano, —N(R^{A})₂, —OR^{A}, —C(O)N(R^{B})₂, —SO₂N(R^{B})₂, —SO₂R^{C}, or -Q-R^{X};
   each R^{X} is independently hydroxyl, optionally substituted C₁₋₆ alkyl, optionally substituted 6- to 10-membered aryl, optionally substituted 3- to 10-membered heterocyclyl, optionally substituted 5- to 10-membered heteroaryl, —N(R^{A})₂, —C(O)N(R^{B})₂, —SO₂N(R^{B})₂, —SO₂R^{C}, or optionally substituted C₁₋₆alkoxy;
   each R^{A} is independently hydrogen, optionally substituted C₁₋₆ alkyl, optionally substituted 6- to 10-membered aryl C₁₋₆ alkyl, optionally substituted C₃₋₈ cycloalkyl, optionally substituted 6- to 10-membered aryl, optionally substituted 3- to 10-membered heterocyclyl, optionally substituted 5- to 10-membered heteroaryl, optionally substituted 5- to 10-membered heteroaryl C₁₋₆ alkyl, or —SO₂R^{C}; or two R^{A}, together with the atom to which they are attached, combine to form an optionally substituted 3- to 10-membered heterocyclyl;
   each R^{B} is independently hydrogen, optionally substituted C₁₋₆ alkyl, optionally substituted C₂₋₆ alkoxy C₁₋₆ alkyl, optionally substituted 6- to 10-membered aryl C₁₋₆ alkyl, optionally substituted C₃₋₈ cycloalkyl, optionally substituted 6- to 10-membered aryl, optionally substituted 3- to 10-membered heterocyclyl, or optionally substituted 5- to 10-membered heteroaryl; or two R^{B}, together with the atom to which they are attached, combine to form an optionally substituted 3- to 10-membered heterocyclyl;
   each R^{C} is independently optionally substituted C₁₋₆ alkyl, optionally substituted C₃₋₈ cycloalkyl, optionally substituted 6- to 10-membered aryl, optionally substituted 3- to 10-membered heterocyclyl, or optionally substituted 5- to 10-membered heteroaryl;
   Q is optionally substituted C₁₋₆ alkylene, optionally substituted C₂₋₆ alkenylene, optionally substituted C₂₋₆ alkynylene, optionally substituted C₃₋₈ cycloalkylene, optionally substituted C₃₋₈ cycloalkenylene, optionally substituted 6- to 10-membered arylene, optionally substituted 3- to 10-membered heterocyclylene, or optionally substituted 5- to 10-membered heteroarylene.

In certain embodiments, Z is hydrogen. In certain embodiments, Z is optionally substituted C₁₋₃ alkyl, optionally substituted C₃₋₅ cycloalkyl, or halogen.

In certain embodiments, each of R³ and R⁴ is independently optionally substituted C₁₋₆ alkyl or halogen. In certain embodiments, each of R³ and R⁴ is independently optionally substituted C₁₋₆ alkenyl, optionally substituted C₁₋₆ alkynyl, or optionally substituted C₃₋₅ cycloalkyl.

Item 3. The compound or a pharmaceutically acceptable salt, stereoisomer, or isotopic variant thereof according to Item 2, wherein the compound is enriched for the atropisomer of Formula (IA) or (IIA):

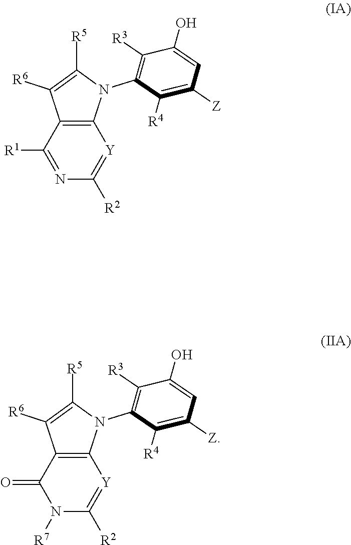

Item 4. The compound or a pharmaceutically acceptable salt, stereoisomer, or isotopic variant thereof according to Item 1, wherein the compound is of Formula (I′) or (II′)

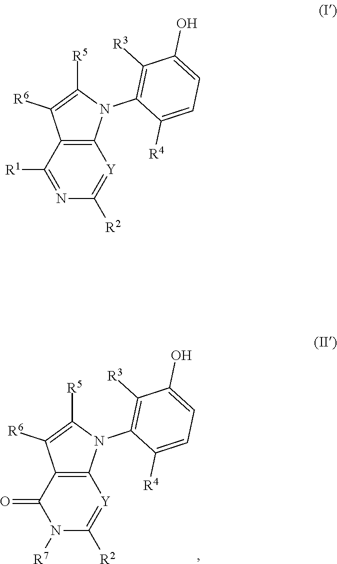

Y is N or CR⁸;
each of R¹, R², and R⁸ is independently hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted 3- to 10-membered heterocyclyl, optionally substituted 3- to 10-membered heterocyclyl C₁₋₆ alkyl, optionally substituted 6- to 10-membered aryl, optionally substituted 5- to 10-membered heteroaryl, optionally substituted 5- to 10-membered heteroaryl C₁₋₆ alkyl, halogen, cyano, —N(R^{A})₂, —OR^{A}, —SR^{A}, —C(O)N(R^{B})₂, —SO₂N(R^{B})₂, —SO₂R^{C}, or -Q-R^{X};
each of R³ and R⁴ is independently optionally substituted C₁₋₆ alkyl, optionally substituted C₁₋₆ alkenyl, optionally substituted C₁₋₆ alkynyl, optionally substituted C₃₋₅ cycloalkyl, or halogen;
R⁵ is H or —N(R^{A})₂;
R⁶ is —C(O)NH(R^{B}), —C(O)R^{C}, or —SO₂R^{C};
R⁷ is hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted 3- to 10-membered heterocyclyl, optionally substituted 3- to 10-membered heterocyclyl C₁₋₆ alkyl, optionally substituted 6- to 10-membered aryl, optionally substituted 5- to 10-membered heteroaryl, optionally substituted 5- to 10-membered heteroaryl C₁₋₆ alkyl, halogen, cyano, —N(R^{A})₂, —OR^{A}, —C(O)N(R^{B})₂, —SO₂N(R^{B})₂, —SO₂R^{C}, or -Q-R^{X};
each R^{X} is independently hydroxyl, optionally substituted C₁₋₆ alkyl, optionally substituted 6- to 10-membered aryl, optionally substituted 3- to 10-membered heterocyclyl, optionally substituted 5- to 10-membered heteroaryl, —N(R^{A})₂, —C(O)N(R^{B})₂, —SO₂N(R^{B})₂, —SO₂R^{C}, or optionally substituted C₁₋₆alkoxy;
each R^{A} is independently hydrogen, optionally substituted C₁₋₆ alkyl, optionally substituted 6- to 10-membered aryl C₁₋₆ alkyl, optionally substituted C₃₋₈ cycloalkyl, optionally substituted 6- to 10-membered aryl, optionally substituted 3- to 10-membered heterocyclyl, optionally substituted 5- to 10-membered heteroaryl, optionally substituted 5- to 10-membered heteroaryl C₁₋₆ alkyl, or —SO₂R^{C}; or two R^{A}, together with the atom to which they are attached, combine to form an optionally substituted 3- to 10-membered heterocyclyl;
each R^{B} is independently hydrogen, optionally substituted C₁₋₆ alkyl, optionally substituted C₂₋₆ alkoxy C₁₋₆ alkyl, optionally substituted 6- to 10-membered aryl C₁₋₆ alkyl, optionally substituted C₃₋₈ cycloalkyl, optionally substituted 6- to 10-membered aryl, optionally substituted 3- to 10-membered heterocyclyl, or optionally substituted 5- to 10-membered heteroaryl; or two R^{B}, together with the atom to which they are attached, combine to form an optionally substituted 3- to 10-membered heterocyclyl;
each R^{C} is independently optionally substituted C₁₋₆ alkyl, optionally substituted C₃₋₈ cycloalkyl, optionally substituted 6- to 10-membered aryl, optionally substituted 3- to 10-membered heterocyclyl, or optionally substituted 5- to 10-membered heteroaryl;
Q is optionally substituted C₁₋₆ alkylene, optionally substituted C₂₋₆ alkenylene, optionally substituted C₂₋₆ alkynylene, optionally substituted C₃₋₈ cycloalkylene, optionally substituted C₃₋₈ cycloalkenylene, optionally substituted 6- to 10-membered arylene, optionally substituted 3- to 10-membered heterocyclylene, or optionally substituted 5- to 10-membered heteroarylene.

In certain embodiments, each of R³ and R⁴ is independently optionally substituted C₁₋₆ alkyl or halogen. In certain embodiments, each of R³ and R⁴ is independently optionally substituted C₁₋₆ alkenyl, optionally substituted C₁₋₆ alkynyl, or optionally substituted C₃₋₅ cycloalkyl.

Item 5. The compound or a pharmaceutically acceptable salt, stereoisomer, or isotopic variant thereof according to Item 4, wherein the compound is enriched for the atropisomer of Formula (IA′) or (IIA′):

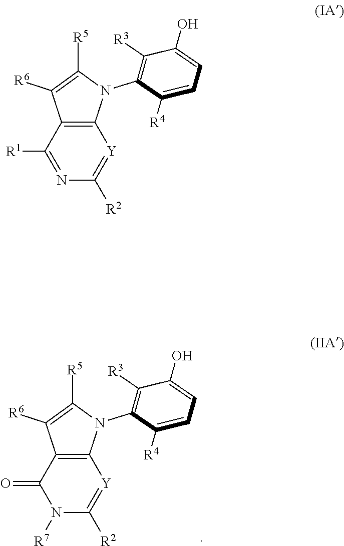

Item 6. The compound or a pharmaceutically acceptable salt, stereoisomer, or isotopic variant thereof according to any one of the preceding Items, wherein Y is N.

Item 7. The compound or a pharmaceutically acceptable salt, stereoisomer, or isotopic variant thereof according to any one of the preceding Items, wherein Y is CR⁸, and R⁸ is hydrogen or optionally substituted C₁₋₆ alkyl.

Item 8. The compound or a pharmaceutically acceptable salt, stereoisomer, or isotopic variant thereof according to any one of the preceding Items, wherein

R¹ is hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted 3- to 10-membered heterocyclyl, optionally substituted 3- to 10-membered heterocyclyl C₁₋₆ alkyl, optionally substituted 6- to 10-membered aryl, optionally substituted 5- to 10-membered heteroaryl, optionally substituted 5- to 10-membered heteroaryl C₁₋₆ alkyl, halogen, cyano, —N(R^{A}′)₂, —OR^{A}′, —SR^{A}′, —C(O)N(R^{B}′)₂, —SO₂N(R^{B}′)₂, or —SO₂R^{C}′, each of R^{A}′, R^{B}′, and R^{C}′ is independently optionally substituted C₁₋₆ alkyl, optionally substituted C₃₋₈ cycloalkyl, optionally substituted 6- to 10-membered aryl, optionally substituted 3- to 10-membered heterocyclyl, or optionally substituted 5- to 10-membered heteroaryl, in which said C₁₋₆ alkyl, C₂₋₆ alkenyl, and C₂₋₆ alkynyl are optionally substituted with one or more substitutes selected from halogen, C₁₋₄ alkyloxy, C₁₋₄ haloalkyloxy, and C₁₋₄ haloalkylmercapto; said C₃₋₈ cycloalkyl and C₃₋₈ cycloalkenyl are optionally substituted with one or more substitutes selected from C₁₋₄ alkyl, halogen, C₁₋₄ alkyloxy, C₁₋₄ haloalkyloxy, and C₁₋₄ haloalkylmercapto; and said heterocyclyl, aryl, and heteroaryl are optionally substituted with one or more substitutes selected from C₁₋₄ alkyl, halogen, C₁₋₄ alkyloxy, C₁₋₄ haloalkyloxy, 4- to 10-membered heterocyclyl, 6- to 10-membered aryl, and 5- to 10-membered heteroaryl.

Item 9. The compound or a pharmaceutically acceptable salt, stereoisomer, or isotopic variant thereof according to any one of the preceding Items, wherein R¹ is hydrogen, C₁₋₆ alkyl, C₃₋₈ cycloalkyl, 3- to 10-membered heterocyclyl, halogen, —OR^{A}′, or —SR^{A}′, in which R^{A}′ is C₁₋₆ alkyl, C₁₋₆ haloalkyl, C₃₋₈ cycloalkyl, optionally substituted 6- to 10-membered aryl, optionally substituted 3- to 10-membered heterocyclyl, or optionally substituted 5- to 10-membered heteroaryl.

Item 10. The compound or a pharmaceutically acceptable salt, stereoisomer, or isotopic variant thereof according to any one of the preceding Items, wherein R¹ is hydrogen, fluoride, methoxy, monofluoromethoxy, difluoromethoxy, trifluoromethoxy, trifluoromethylmercapto, —OCD₃,

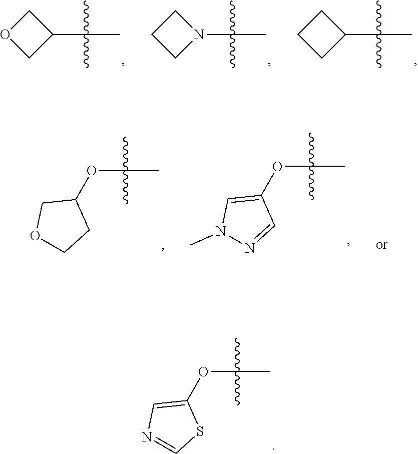

Item 11. The compound or a pharmaceutically acceptable salt, stereoisomer, or isotopic variant thereof according to any one of the preceding Items, wherein R¹ is hydrogen.

Item 12. The compound or a pharmaceutically acceptable salt, stereoisomer, or isotopic variant thereof according to any one of the preceding Items, wherein R² is hydrogen, C₁₋₆ alkyl, C₃₋₈ cycloalkyl, or 3- to 10-membered heterocyclyl, in which said C₁₋₆ alkyl, C₃₋₈ cycloalkyl, and 3- to 10-membered heterocyclyl are optionally substituted by one, two, three, four, or five groups independently selected from the group consisting of halogen and C₁₋₆ alkoxy, as valency allows.

Item 13. The compound or a pharmaceutically acceptable salt, stereoisomer, or isotopic variant thereof according to any one of the preceding Items, wherein R² is methyl or trifluoromethyl.

Item 14. The compound or a pharmaceutically acceptable salt, stereoisomer, or isotopic variant thereof according to any one of the preceding Items, wherein R³ is C₁₋₆ alkyl, particularly methyl; or R³ is halogen, particularly chloride.

Item 15. The compound or a pharmaceutically acceptable salt, stereoisomer, or isotopic variant thereof according to any one of the preceding Items, wherein R⁴ is C₁₋₆ alkyl, particularly methyl.

Item 16. The compound or a pharmaceutically acceptable salt, stereoisomer, or isotopic variant thereof according to any one of the preceding Items, wherein R⁵ is —N(R^{A})₂.

Item 17. The compound or a pharmaceutically acceptable salt, stereoisomer, or isotopic variant thereof according to any one of the preceding Items, wherein R⁵ is —NH₂.

Item 18. The compound or a pharmaceutically acceptable salt, stereoisomer, or isotopic variant thereof according to any one of the preceding Items, wherein R⁶ is —C(O)NH(R^{B}).

Item 19. The compound or a pharmaceutically acceptable salt, stereoisomer, or isotopic variant thereof according to any one of the preceding Items, wherein R⁶ is —C(O)NH₂ or —C(O)NH(Me).

Item 20. The compound or a pharmaceutically acceptable salt, stereoisomer, or isotopic variant thereof according to any one of the preceding Items, wherein R⁶ is —C(O)NH₂.

Item 21. The compound or a pharmaceutically acceptable salt, stereoisomer, or isotopic variant thereof according to any one of the preceding Items, wherein R⁷ is hydrogen, optionally substituted C₁₋₆ alkyl, optionally substituted C₂₋₆ alkenyl, optionally substituted C₃₋₈ cycloalkyl, optionally substituted C₃₋₈ cycloalkenyl, optionally substituted 3- to 10-membered heterocyclyl, optionally substituted 6- to 10-membered aryl, or optionally substituted 5- to 10-membered heteroaryl.

Item 22. The compound or a pharmaceutically acceptable salt, stereoisomer, or isotopic variant thereof according to any one of the preceding Items, wherein R⁷ is hydrogen, optionally substituted C₁₋₆ alkyl, C₃₋₈ cycloalkyl, 3- to 10-membered heterocyclyl, 6- to 10-membered aryl, or 5- to 10-membered heteroaryl, in which said C₃₋₈ cycloalkyl, 3- to 10-membered heterocyclyl, 6- to 10-membered aryl, and 5- to 10-membered heteroaryl are optionally substituted by C₁₋₆ alkyl.

Item 23. The compound or a pharmaceutically acceptable salt, stereoisomer, or isotopic variant thereof according to any one of the preceding Items, wherein R⁷ is methyl, fluoromethyl, cyclopropyl, or 1-methyl-1H-pyrazol-4-yl.

Item 24. The compound or a pharmaceutically acceptable salt, stereoisomer, or isotopic variant thereof according to any one of the preceding Items, wherein the isotopic variant is a deuterated variant.

Item 25. The compound or a pharmaceutically acceptable salt, stereoisomer, or isotopic variant thereof according to Item 1, wherein the compound is:

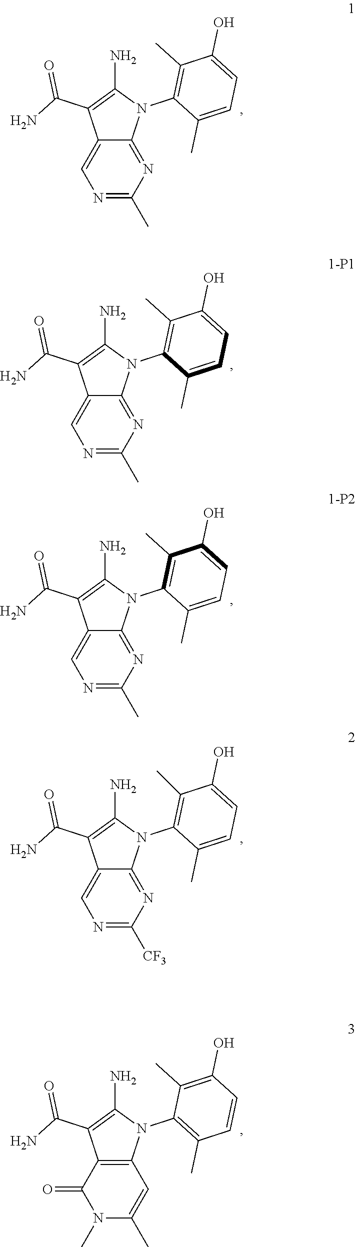

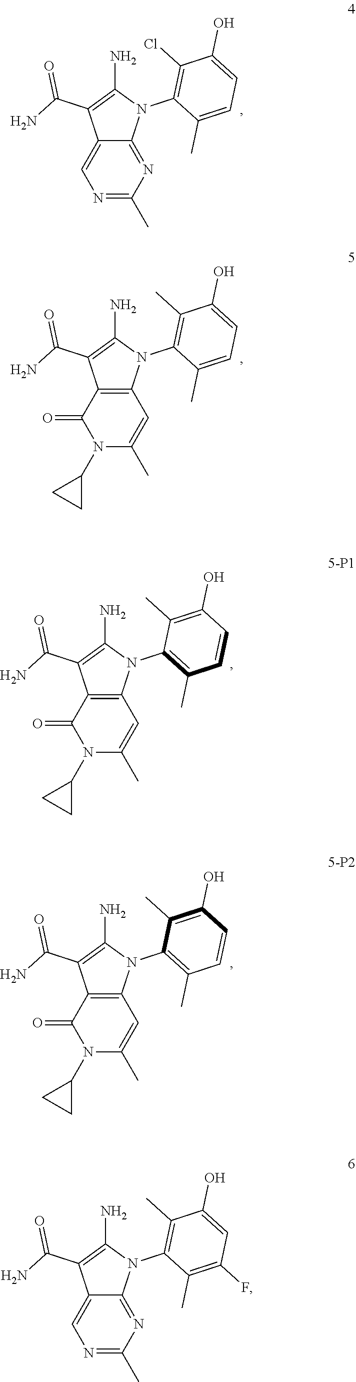

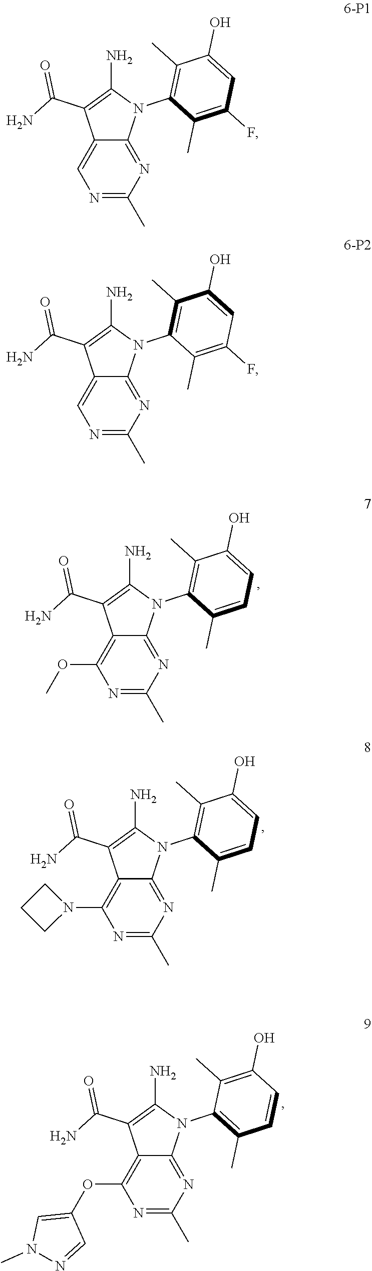

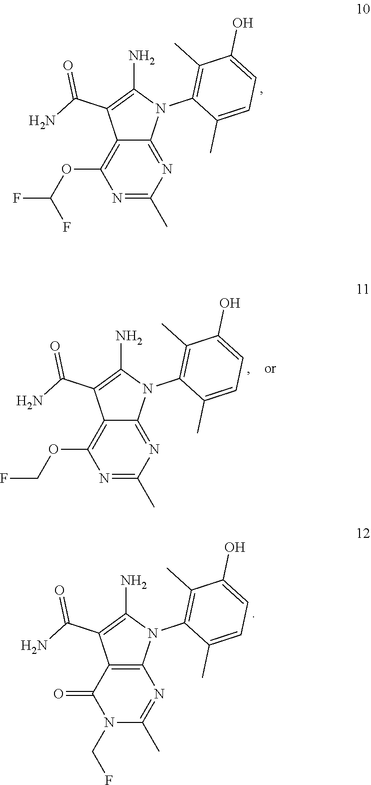 [SMILES: Cc1nc(OC(F)F)c2c(C(N)=O)c(N)n(-c3c(C)ccc(O)c3C)c2n1 ; Cc1nc(OCF)c2c(C(N)=O)c(N)n(-c3c(C)ccc(O)c3C)c2n1 ; Cc1ccc(O)c(C)c1-n1c(N)c(C(N)=O)c2c(=O)n(CF)c(C)nc21]

Item 26. The compound or a pharmaceutically acceptable salt, stereoisomer, or isotopic variant thereof according to Item 1, wherein the compound is:

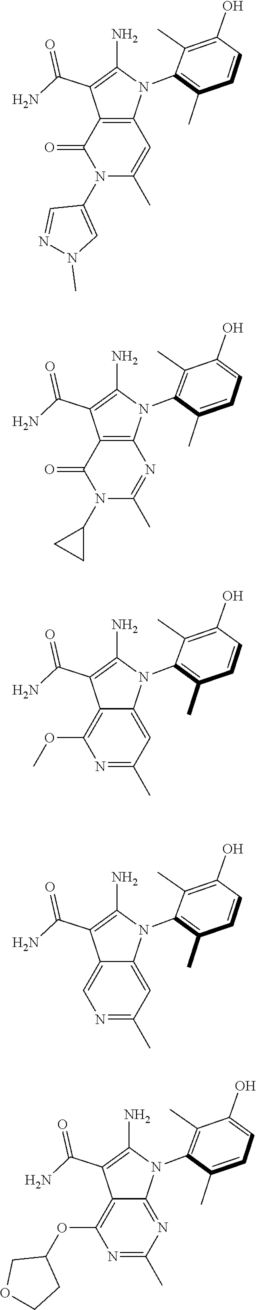

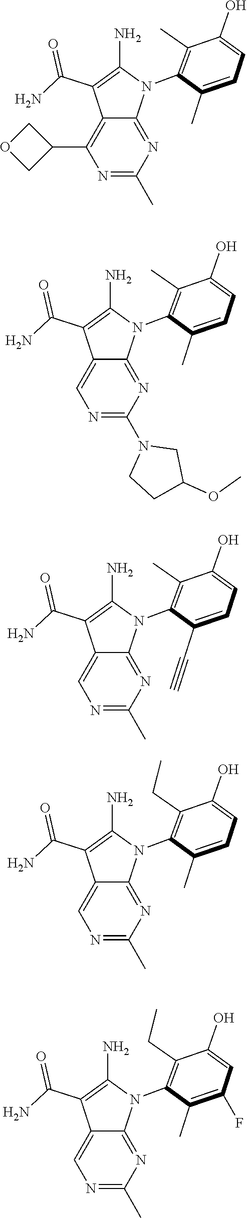

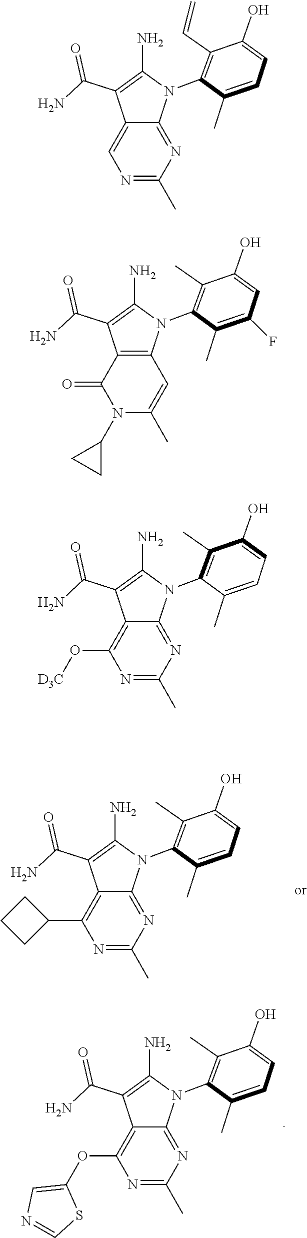

Item 27. A pharmaceutical composition for treating a PKMYT1-associated disease or condition, which comprises the compound or a pharmaceutically acceptable salt, stereoisomer, or isotopic variant thereof according to any one of Items 1 to 26, and a pharmaceutically acceptable carrier or excipient.

Item 28. The pharmaceutical composition according to Item 27, wherein the PKMYT1-associated disease or condition is tumor, and particularly, advanced solid tumor.

Item 29. The pharmaceutical composition according to Item 27, wherein the PKMYT1-associated disease or condition is selected from the group consisting of uterine cancer, ovarian cancer, breast cancer, gastric cancer, esophageal cancer, lung cancer (e.g., non-small-cell lung cancer), endometrial cancer, colorectal cancer, hepatocellular carcinoma, glioblastoma, and neuroblastoma.

Item 30. A method of treating a PKMYT1-associated disease or condition in a subject in need thereof, which comprises administering to the subject a therapeutically effective amount of the compound or a pharmaceutically acceptable salt, stereoisomer, or isotopic variant thereof according to any one of Items 1 to 26.

Item 31. The method according to Item 30, wherein the PKMYT1-associated disease or condition is tumor, and particularly, advanced solid tumor.

Item 32. The method according to Item 30, wherein the PKMYT1-associated disease or condition is selected from the group consisting of uterine cancer, ovarian cancer, breast cancer, gastric cancer, esophageal cancer, lung cancer (e.g., non-small-cell lung cancer), endometrial cancer, colorectal cancer, hepatocellular carcinoma, glioblastoma, and neuroblastoma.

Item 33. The compound or a pharmaceutically acceptable salt, stereoisomer, or isotopic variant thereof according to any one of Items 1 to 26 for use in the treatment of a PKMYT1-associated disease or condition.

Item 34. The compound or a pharmaceutically acceptable salt, stereoisomer, or isotopic variant thereof according to Item 33, wherein the PKMYT1-associated disease or condition is tumor, and particularly, advanced solid tumor.

Item 35. The compound or a pharmaceutically acceptable salt, stereoisomer, or isotopic variant thereof according to Item 33, wherein the PKMYT1-associated disease or condition is selected from the group consisting of uterine cancer, ovarian cancer, breast cancer, gastric cancer, esophageal cancer, lung cancer (e.g., non-small-cell lung cancer), endometrial cancer, colorectal cancer, hepatocellular carcinoma, glioblastoma, and neuroblastoma.

Item 36. Use of a compound or a pharmaceutically acceptable salt, stereoisomer, or isotopic variant thereof according to any one of Items 1 to 26 in the manufacture of a medicament for treating a PKMYT1-associated disease or condition.

Item 37. The use according to Item 36, wherein the PKMYT1-associated disease or condition is tumor, and particularly, advanced solid tumor.

Item 38. The use according to Item 36, wherein the PKMYT1-associated disease or condition is selected from the group consisting of uterine cancer, ovarian cancer, breast cancer, gastric cancer, esophageal cancer, lung cancer (e.g., non-small-cell lung cancer), endometrial cancer, colorectal cancer, hepatocellular carcinoma, glioblastoma, and neuroblastoma.

Item 39. A kit for treating a PKMYT1-associated disease or condition, which comprises a compound or a pharmaceutically acceptable salt, stereoisomer, or isotopic variant thereof according to any one of Items 1 to 26, a container, and optionally a package insert or label indicating treatment of said disease or condition.

Item 40. The kit according to Item 39, wherein the PKMYT1-associated disease or condition is tumor, and particularly, advanced solid tumor.

Item 41. The kit according to Item 39, wherein the PKMYT1-associated disease or condition is selected from the group consisting of uterine cancer, ovarian cancer, breast cancer, gastric cancer, esophageal cancer, lung cancer (e.g., non-small-cell lung cancer), endometrial cancer, colorectal cancer, hepatocellular carcinoma, glioblastoma, and neuroblastoma.

### Definitions

The terms used but not defined herein have their ordinary meaning and the meaning of such terms is independent at each occurrence thereof. Nevertheless, unless otherwise stated, the following definitions apply throughout the specification and claims.

As used herein, the singular forms “a”, “an”, and “the” include plural referents unless expressly stated to the contrary.

As used herein, the terms “comprise” and “include” are intended to specify the presence of stated features, integers, components, or steps, but they do not preclude the presence or addition of one or more other features, integers, components, steps, or groups thereof.

Definitions of specific functional groups and chemical terms are described in more detail below. For purpose of this disclosure, the chemical elements are identified in accordance with the Periodic Table of the Elements, CAS version, Handbook of Chemistry and Physics, 75^{th} Edition, inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modem Methods of Organic Synthesis, 3^{rd} Edition, Cambridge University Press, Cambridge, 1987.

All ranges cited herein are inclusive, unless expressly stated to the contrary.

When a range of values is listed, it is intended to encompass each value and sub-range within the range. For example, “C₁₋₆” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆. For example, a heteroaromatic ring described as containing from “1 to 4 heteroatoms” means that the ring can contain 1, 2, 3 or 4 heteroatoms. It is also to be understood that any range cited herein includes within its scope all of the sub-ranges within that range. Thus, for example, a heterocyclic ring described as containing from “1 to 4 heteroatoms” is intended to include as aspects thereof, heterocyclic rings containing 2 to 4 heteroatoms, 3 or 4 heteroatoms, 1 to 3 heteroatoms, 2 or 3 heteroatoms, 1 or 2 heteroatoms, 1 heteroatom, 2 heteroatoms, 3 heteroatoms, or 4 heteroatoms.

When any variable occurs more than one time in any constituent or in Formula (I) or (II) or in any other formula depicting and describing the compounds of the present disclosure, its definition at each occurrence is independent of its definition at every other occurrence. Also, combinations of substituents and/or variables are permissible only if such combinations result in stable compounds.

As used herein, the term “alkyl” refers to a linear or branched chain saturated hydrocarbon group. The term “Cᵢ₋ⱼ alkyl” refers to an alkyl having i to j carbon atoms. Alkyl groups may contain 1 to 10 carbon atoms, unless otherwise stated. In certain embodiments, alkyl groups contain 1 to 6 carbon atoms (C₁₋₆), such as, 1 to 5 carbon atoms (C₁₋₅), 1 to 4 carbon atoms (C₁₋₄), 1 to 3 carbon atoms (C₁₋₃), or 1 to 2 carbon atoms (C₁₋₂). Non-limiting examples of alkyl groups include methyl, ethyl, n- and iso-propyl, n-, sec-, iso-, and tert-butyl, neopentyl, and the like. Alkyl groups may be optionally substituted (i.e., unsubstituted or substituted), as valency permits, with one, two, three, or, in the case of alkyl groups of two carbons or more, four or more substituents independently selected from the group consisting of: amino; alkoxy; aryl; aryloxy; azido; cycloalkyl; cycloalkyloxy; cycloalkenyl; cycloalkynyl; halogen; heterocyclyl; (heterocyclyl)oxy; heteroaryl; hydroxy; nitro; thiol; silyl; cyano; alkylmercapto; alkylsulfonyl; alkylsulfinyl; alkylsulfenyl; ═O; ═S; —C(O)R or —SO₂R, in which R is amino; and ═NR′, in which R′ is H, alkyl, aryl, or heterocyclyl. Each of the substituents may itself be unsubstituted or, as valency permits, substituted with unsubstituted substituent(s) defined herein for each respective group. In certain embodiments, alkyl groups may be optionally substituted with one or more substitutes selected from halogen, C₁₋₄ alkyloxy, C₁₋₄ haloalkyloxy, and C₁₋₄ haloalkylmercapto.

As used herein, the term “alkylene” refers to a divalent substituent that is a monovalent alkyl having one hydrogen atom replaced with a valency. Alkylene groups may be unsubstituted or substituted. An optionally substituted alkylene is an alkylene that is optionally substituted as described herein for alkyl.

As used herein, the term “alkenyl” refers to a linear or branched-chain hydrocarbon radical having at least one (such as one, two, or three) carbon-carbon double bond, which may be optionally substituted (i.e., unsubstituted or substituted) independently with one or more substituents described herein, and includes radicals having “cis” and “trans” orientations, or alternatively, “E” and “Z” orientations. Alkenyl groups may contain 2 to 10 carbon atoms, unless otherwise stated. In certain embodiments, alkenyl groups may contain 2 to 6 carbon atoms, such as 2 to 5 carbon atoms, 2 to 4 carbon atoms, 2 to 3 carbon atoms. In certain embodiments, alkenyl groups contain 2 carbon atoms. Non-limiting examples of alkenyl groups include ethylenyl (vinyl), propenyl, butenyl, pentenyl, 1-methyl-2-buten-1-yl, 5-hexenyl, etc. An optionally substituted alkenyl is an alkenyl that is optionally substituted as described herein for alkyl.

As used herein, the term “alkenylene” refers to a divalent substituent that is a monovalent alkenyl having one hydrogen atom replaced with a valency. Alkenylene groups may be unsubstituted or substituted. An optionally substituted alkenylene is an alkenylene that is optionally substituted as described herein for alkyl.

As used herein, the term “alkynyl” refers to a linear or branched hydrocarbon radical having at least one (such as one, two, or three) carbon-carbon triple bond, which may be optionally substituted (i.e., unsubstituted or substituted) independently with one or more substituents described herein. Alkynyl groups may contain 2 to 10 carbon atoms, unless otherwise stated. In certain embodiments, alkynyl groups may contain 2 to 6 carbon atoms, such as 2 to 5 carbon atoms, 2 to 4 carbon atoms, 2 to 3 carbon atoms. In certain embodiments, alkynyl groups contain 2 carbon atoms. Non-limiting examples of alkynyl groups include ethynyl, 1-propynyl, 2-propynyl, etc. An optionally substituted alkynyl is an alkynyl that is optionally substituted as described herein for alkyl.

As used herein, the term “alkynylene” refers to a divalent substituent that is a monovalent alkynyl having one hydrogen atom replaced with a valency. Alkynylene groups may be unsubstituted or substituted. An optionally substituted alkynylene is an alkynylene that is optionally substituted as described herein for alkyl.

As used herein, the term “cycloalkyl” refers to a monovalent non-aromatic, saturated monocyclic and polycyclic ring system, in which all the ring atoms are carbons and which contains at least three ring forming carbon atoms. Cycloalkyl groups may contain 3 to 10 ring forming carbon atoms, unless otherwise stated. In certain embodiments, cycloalkyl groups may contain 3 to 8 ring forming carbon atoms, such as 3 to 7 ring forming carbon atoms, 3 to 6 ring forming carbon atoms, 3 to 5 ring forming carbon atoms, 3 to 4 ring forming carbon atoms, 3 ring forming carbon atoms, 4 ring forming carbon atoms, 5 ring forming carbon atoms, 6 ring forming carbon atoms, 7 ring forming carbon atoms, 8 ring forming carbon atoms, etc. Particularly, cycloalkyl groups may be monocyclic or bicyclic. Alternatively, bicyclic cycloalkyl groups may include fused, spiro, and bridged cycloalkyl structures. Non-limiting examples of cycloalkyl groups include cyclopropyl, cyclobutyl, cyclopentyl, cyclohexyl, cycloheptyl, 1-bicyclo[2.2.1.]heptyl, 2-bicyclo[2.2.1.]heptyl, 5-bicyclo[2.2.1.]heptyl, 7-bicyclo[2.2.1.]heptyl, and decalinyl. The cycloalkyl group may be optionally substituted (i.e., unsubstituted or substituted) with one, two, three, four, or five substituents independently selected from the group consisting of: alkyl; alkenyl; alkynyl; alkoxy; alkylmercapto; alkylsulfinyl; alkylsulfenyl; alkylsulfonyl; amino; aryl; aryloxy; azido; cycloalkyl; cycloalkyloxy; cycloalkenyl; cycloalkynyl; halogen; heteroalkyl; heteroalkenyl; heteroalkynyl; heterocyclyl; (heterocyclyl)oxy; heteroaryl; hydroxy; nitro; thiol; silyl; cyano; ═O; ═S; —SO₂R, in which R is optionally substituted amino; ═NR′, in which R′ is H, alkyl, aryl, or heterocyclyl; and —CON(R″)₂, in which each R″ is independently H or alkyl, or both R″, together with the atom to which they are attached, combine to form heterocyclyl. Each of the substituents may itself be unsubstituted or substituted with unsubstituted substituent(s) defined herein for each respective group. In certain embodiments, cycloalkyl groups may be optionally substituted with one or more substitutes selected from C₁₋₄ alkyl, halogen, C₁₋₄ alkyloxy, C₁₋₄ haloalkyloxy, and C₁₋₄ haloalkylmercapto.

As used herein, the term “cycloalkylene” refers to a divalent substituent that is a cycloalkyl having one hydrogen atom replaced with a valency. Cycloalkylene groups may be unsubstituted or substituted. An optionally substituted cycloalkylene is a cycloalkylene that is optionally substituted as described herein for cycloalkyl.

As used herein, the term “cycloalkenyl” refers to a non-aromatic carbocyclic group having at least one (such as one, two, or three) double bond in the ring. Cycloalkenyl groups may contain 3 to 10 ring forming carbon atoms, unless otherwise stated. In certain embodiments, cycloalkenyl groups may contain 3 to 8 ring forming carbon atoms, such as 3 to 7 ring forming carbon atoms, 3 to 6 ring forming carbon atoms, 3 to 5 ring forming carbon atoms, 3 to 4 ring forming carbon atoms, 3 ring forming carbon atoms, 4 ring forming carbon atoms, 5 ring forming carbon atoms, 6 ring forming carbon atoms, 7 ring forming carbon atoms, 8 ring forming carbon atoms, etc. Non-limiting examples of cycloalkenyl groups include cycloprop-1-enyl, cycloprop-2-enyl, cyclobut-1-enyl, cyclobut-1-enyl, cyclobut-2-enyl, cyclopent-1-enyl, cyclopent-2-enyl, cyclopent-3-enyl, norbornen-1-yl, norbornen-2-yl, norbornen-5-yl, and norbornen-7-yl. Cycloalkenyl groups may be unsubstituted or substituted. An optionally substituted cycloalkenyl is a cycloalkenyl that is optionally substituted as described herein for cycloalkyl.

As used herein, the term “cycloalkenylene” refers to a divalent substituent that is a cycloalkenyl having one hydrogen atom replaced with a valency. Cycloalkenylene groups may be unsubstituted or substituted. An optionally substituted cycloalkenylene is a cycloalkenylene that is optionally substituted as described herein for cycloalkyl.

As used herein, the term “cycloalkynyl” refers to a monovalent carbocyclic group having at least one (such as one, two, or three) carbon-carbon triple bonds. Cycloalkynyl groups may contain 3 to 10 ring forming carbon atoms, unless otherwise stated. In certain embodiments, cycloalkynyl groups may contain 3 to 8 ring forming carbon atoms, such as 3 to 7 ring forming carbon atoms, 3 to 6 ring forming carbon atoms, 3 to 5 ring forming carbon atoms, 3 to 4 ring forming carbon atoms, 3 ring forming carbon atoms, 4 ring forming carbon atoms, 5 ring forming carbon atoms, 6 ring forming carbon atoms, 7 ring forming carbon atoms, 8 ring forming carbon atoms, etc. Cycloalkynyl groups may include one transannular bond or bridge. Non-limiting examples of cycloalkynyl groups include cyclooctynyl, cyclononynyl, cyclodecynyl, and cyclodecadiynyl. Cycloalkynyl groups may be unsubstituted or substituted. An optionally substituted cycloalkynyl is a cycloalkynyl that is optionally substituted as described herein for cycloalkyl.

As used herein, the term “heterocyclyl” refers to a monocyclic, bicyclic, tricyclic, or tetracyclic ring system having fused, bridged, and/or spiro 3- to 10-membered rings, unless otherwise stated, containing one, two, three, or four heteroatoms independently selected from the group consisting of nitrogen, oxygen, and sulfur as ring forming atoms. In certain embodiments, heterocyclyl groups may be 3-, 4-, 5-, 6-, 7-, 8-, 9-, or 10-membered. In certain embodiments, heterocyclyl groups may be 3- to 9-membered, 3- to 8-membered, 3- to 6-membered, 4- to 10-membered, 4- to 8-membered, 4- to 6-membered, or 5- to 8-membered. In certain embodiments, heterocyclyl groups may contain one, two, or three heteroatoms. In certain embodiments, heterocyclyl may be a monocyclic, bicyclic, tricyclic, or tetracyclic ring system having fused or bridged 5-, 6-, 7-, or 8-membered rings, containing one, two, three, or four heteroatoms independently selected from the group consisting of nitrogen, oxygen, and sulfur. Heterocyclyl can be aromatic or non-aromatic. In certain embodiments, heterocyclyl is non-aromatic. In certain embodiments, non-aromatic 5-membered heterocyclyl has zero or one double bonds, non-aromatic 6- and 7-membered heterocyclyl groups have zero to two double bonds, and non-aromatic 8-membered heterocyclyl groups have zero to two double bonds and/or zero or one carbon-carbon triple bond. In certain embodiments, heterocyclyl is a saturated ring. In certain embodiments, heterocyclyl groups may include up to 9 carbon atoms. Non-aromatic heterocyclyl groups include pyrrolinyl, pyrrolidinyl, pyrazolinyl, pyrazolidinyl, imidazolinyl, imidazolidinyl, piperidinyl, homopiperidinyl, piperazinyl, pyridazinyl, oxazolidinyl, isoxazolidinyl, morpholinyl, thiomorpholinyl, thiazolidinyl, isothiazolidinyl, thiazolidinyl, tetrahydrofuranyl, dihydrofuranyl, tetrahydrothienyl, dihydrothienyl, dihydroindolyl, tetrahydroquinolyl, tetrahydroisoquinolyl, pyranyl, dihydropyranyl, dithiazolyl, etc. If the heterocyclic ring system has at least one aromatic resonance structure or at least one aromatic tautomer, such structure is an aromatic heterocyclyl (i.e., heteroaryl). Non-limiting examples of heteroaryl groups include benzimidazolyl, benzofuryl, benzothiazolyl, benzothienyl, benzoxazolyl, furyl, imidazolyl, indolyl, isoindazolyl, isoquinolinyl, isothiazolyl, isothiazolyl, isoxazolyl, oxadiazolyl, oxazolyl, purinyl, pyrrolyl, pyridinyl, pyrazinyl, pyrimidinyl, quinazolinyl, quinolinyl, thiadiazolyl (e.g., 1,3,4-thiadiazole), thiazolyl, thienyl, triazolyl, tetrazolyl, etc. The term “heterocyclyl” also includes a heterocyclic compound having a bridged multicyclic structure in which one or more carbons and/or heteroatoms bridges two non-adjacent members of a monocyclic ring, e.g., quinuclidine, tropanes, or diaza-bicyclo[2.2.2]octane. The term “heterocyclyl” includes bicyclic, tricyclic, and tetracyclic groups in which any of the above heterocyclic rings is fused to one, two, or three carbocyclic rings, e.g., an aryl ring, a cyclohexane ring, a cyclohexene ring, a cyclopentane ring, a cyclopentene ring, or another monocyclic heterocyclic ring. Examples of fused heterocyclyl groups include 1,2,3,5,8,8a-hexahydroindolizine; 2,3-dihydrobenzofuran; 2,3-dihydroindole; and 2,3-dihydrobenzothiophene. The heterocyclyl group may be unsubstituted or substituted with one, two, three, four or five substituents independently selected from the group consisting of: alkyl; alkenyl; alkynyl; alkoxy; alkylsulfinyl; alkylsulfenyl; alkylsulfonyl; amino; aryl; aryloxy; azido; cycloalkyl; cycloalkoxy; cycloalkenyl; cycloalkynyl; halogen; heteroalkyl; heterocyclyl; (heterocyclyl)oxy; heteroaryl; hydroxy; nitro; thiol; silyl; cyano; —C(O)R or —SO₂R, where R is amino or alkyl; ═O; S; ═NR′, where R′ is H, alkyl, aryl, or heterocyclyl. Each of the substituents may itself be unsubstituted or substituted with unsubstituted substituent(s) defined herein for each respective group. In certain embodiments, heterocyclyl groups may be optionally substituted with one or more substitutes selected from 4- to 10-membered heterocyclyl, 6- to 10-membered aryl, and 5- to 10-membered heteroaryl.

As used herein, the term “heterocyclylene” refers to a divalent substituent that is an heterocyclyl having one hydrogen atom replaced with a valency. Heterocyclylene groups may be unsubstituted or substituted. An optionally substituted heterocyclylene is an heterocyclylene that is optionally substituted as described herein for heterocyclyl.

As used herein, the term “aryl” refers to a mono-, bicyclic, or multicyclic carbocyclic ring system having at least one aromatic rings. Aryl groups may be 6- to 10-membered, unless otherwise stated. In certain embodiments, aryl groups may contain 6 ring forming carbon atoms. All ring forming atoms within a carbocyclic aryl group are carbon atoms. Non-limiting examples of aryl groups include phenyl, naphthyl, 1,2-dihydronaphthyl, 1,2,3,4-tetrahydronaphthyl, fluorenyl, indanyl, indenyl, etc. In certain embodiments, aryl is phenyl or naphthyl. In certain embodiments, aryl is phenyl. In the context of the present specification, the terms “aryl” and “aromatic ring” may be used interchangeably. Aryl groups may be unsubstituted or substituted. An optionally substituted aryl group may be an aryl optionally substituted with one, two, three, four, or five substituents independently selected from the group consisting of: alkyl; alkenyl; alkynyl; alkoxy; alkylsulfinyl; alkylsulfenyl; alkylsulfonyl; amino; aryl; aryloxy; azido; cycloalkyl; cycloalkoxy; cycloalkenyl; cycloalkynyl; halogen; heteroalkyl; heteroalkenyl; heteroalkynyl; heterocyclyl; (heterocyclyl)oxy; heteroaryl; hydroxy; nitro; thiol; silyl; —(CH₂)ₙ—C(O)OR′; —C(O)R; and —SO₂R, in which R is amino or alkyl, R′ is H or alkyl, and n is 0 or 1. Each of the substituents may itself be unsubstituted or substituted with unsubstituted substituent(s) defined herein for each respective group. In certain embodiments, aryl groups may be optionally substituted with one or more substitutes selected from 4- to 10-membered heterocyclyl, 6- to 10-membered aryl, and 5- to 10-membered heteroaryl.

As used herein, the term “arylene” refers to a divalent substituent that is an aryl having one hydrogen atom replaced with a valency. Arylene groups may be unsubstituted or substituted. An optionally substituted arylene is an arylene that is optionally substituted as described herein for aryl.

As used herein, the term “heteroaryl” refers to a monocyclic ring system, or a fused or bridged bicyclic ring system, in which the ring system contains one, two, three, or four heteroatoms independently selected from the group consisting of nitrogen, oxygen, and sulfur; and at least one of the rings is an aromatic ring. Heteroaryl groups may be 5- to 10-membered, unless otherwise stated. In certain embodiments, heteroaryl groups may be a 5- to 6-membered heteroaryl ring having 1 to 3 heteroatoms independently selected from nitrogen, oxygen, and sulfur; or an 8- to 10-membered bicyclic heteroaryl ring having 1 to 4 heteroatoms independently selected from nitrogen, oxygen, and sulfur. In certain embodiments, heteroaryl groups may contain one, two, or three heteroatoms. In certain embodiments, heteroaryl groups may contain one or two heteroatoms. Non-limiting examples of heteroaryl groups include benzimidazolyl, benzofuryl, benzothiazolyl, benzothienyl, benzoxazolyl, furyl, imidazolyl, indolyl, isoindazolyl, isoquinolinyl, isothiazolyl, isothiazolyl, isoxazolyl, oxadiazolyl, oxazolyl, purinyl, pyrrolyl, pyridinyl, pyrazinyl, pyrimidinyl, quinazolinyl, quinolinyl, thiadiazolyl, thiazolyl, thienyl, triazolyl, tetrazolyl, dihydroindolyl, tetrahydroquinolyl, tetrahydroisoquinolyl, etc. Heteroaryl groups include at least one ring having at least one heteroatom as described above and at least one aromatic ring. For example, a ring having at least one heteroatom may be fused to one, two, or three carbocyclic rings, e.g., an aryl ring, a cyclohexane ring, a cyclohexene ring, a cyclopentane ring, a cyclopentene ring, or another monocyclic heterocyclic ring. Non-limiting examples of fused heteroaryl groups include 1,2,3,5,8,8a-hexahydroindolizine, 2,3-dihydrobenzofuran, 2,3-dihydroindole, 2,3-dihydrobenzothiophene, etc. In the context of the present disclosure, the terms “heteroaryl” and “heteroaromatic ring” may be used interchangeably. Heteroaryl groups may be unsubstituted or substituted. An optionally substituted heteroaryl group may be a heteroaryl optionally substituted with one, two, three, four, or five substituents independently selected from the group consisting of: alkyl; alkenyl; alkynyl; alkoxy; alkylsulfinyl; alkylsulfenyl; alkylsulfonyl; amino; aryl; aryloxy; azido; cycloalkyl; cycloalkoxy; cycloalkenyl; cycloalkynyl; halogen; heteroalkyl; heteroalkenyl; heteroalkynyl; heterocyclyl; (heterocyclyl)oxy; heteroaryl; hydroxy; nitro; thiol; silyl; —(CH₂)ₙ—C(O)OR′; —C(O)R; and —SO₂R, in which R is amino or alkyl, R′ is H or alkyl, and n is 0 or 1. Each of the substituents may itself be unsubstituted or substituted with unsubstituted substituent(s) defined herein for each respective group. In certain embodiments, heteroaryl groups may be optionally substituted with one or more substitutes selected from 4- to 10-membered heterocyclyl, 6- to 10-membered aryl, and 5- to 10-membered heteroaryl.

As used herein, the term “heteroarylene” refers to a divalent substituent that is a heteroaryl having one hydrogen atom replaced with a valency. Heteroarylene groups may be unsubstituted or substituted. An optionally substituted heteroarylene is a heteroarylene that is optionally substituted as described herein for heteroaryl.

As used herein, the term “heteroalkyl” refers to an alkyl as described herein, at least one of the carbon atoms of which is replaced with a heteroatom selected from N, O, and S. The heteroalkyl may be a carbon radical or heteroatom radical (i.e., the heteroatom may appear in the middle or at the end of the radical), and may be optionally substituted independently with one or more substituents described herein for alkyl. The term “heteroalkyl” encompasses alkoxy and heteroalkoxy radicals.

As used herein, the term “heteroalkenyl” refers to an alkenyl as described herein, at least one of the carbon atoms of which is replaced with a heteroatom selected from N, O, and S. The heteroalkenyl may be a carbon radical or heteroatom radical (i.e., the heteroatom may appear in the middle or at the end of the radical), and may be optionally substituted independently with one or more substituents described herein for alkyl.

As used herein, the term “heteroalkynyl” refers to an alkynyl as described herein, at least one of the carbon atoms of which is replaced with a heteroatom selected from N, O, and S. The heteroalkynyl may be a carbon radical or heteroatom radical (i.e., the heteroatom may appear in the middle or at the end of the radical), and may be optionally substituted independently with one or more substituents described herein for alkyl.

As used herein, the term “heteroatom” refers to nitrogen, oxygen, or sulfur, and may include any oxidized form of nitrogen or sulfur, and any quaternized form of a basic nitrogen.

As used herein, the term “oxo” refers to a divalent oxygen atom and the structure of oxo may be shown as ═O.

As used herein, the term “halogen” (or “halo”) refers to fluoride, chloride, bromide, and iodide. In certain embodiments, non-limiting examples of halogen include fluoride, chloride, and bromide. In certain embodiments, halogen is chloride or bromide. In certain embodiments, halogen is fluoride.

As used herein, the term “haloalkyl” refers to an alkyl group as described herein in which one or more of hydrogen atoms have been replaced with one or more halogen atoms selected from the group consisting of fluoride, chloride, bromide, and iodide. Non-limiting examples of haloalkyl groups include —CH₂F, —CHF₂, —CF₃, —CH₂CF₃, —CF₂CF₃, etc. In certain embodiments, haloalkyl groups may be perhaloalkyl groups, such as perfluoroalkyl.

As used herein, the term “arylalkyl” refers to an alkyl group substituted with an aryl group. The aryl and alkyl portions may be optionally substituted as the individual groups described herein.

As used herein, the term “heteroarylalkyl” refers to an alkyl group substituted with a heteroaryl group. The heteroaryl and alkyl portions may be optionally substituted as the individual groups described herein.

As used herein, the term “heterocyclylalkyl” refers to an alkyl group substituted with a heterocyclyl group. The heterocyclyl and alkyl portions may be optionally substituted as the individual groups described herein.

As used herein, the term “(heterocyclyl)oxy” refers to a chemical substituent of formula —OR, in which R is a heterocyclyl group, unless otherwise stated. An (heterocyclyl)oxy group may be optionally substituted as described herein for heterocyclyl.

As used herein, the term “substituted”, when refers to a chemical group, means that the chemical group has one or more hydrogen atoms that is/are removed and replaced by substituents. The term “substituent” as used herein has the ordinary meaning known in the art and refers to a chemical moiety that is covalently attached to, or if appropriate, fused to, a parent group. It is to be understood that substitution at a given atom is limited by valency. It is understood that the substituent can be further substituted.

As used herein, the term “optionally substituted” means that the chemical group may have no substituents (i.e., unsubstituted) or may have one or more substituents (i.e., substituted). It is to be understood that substitution at a given atom is limited by valency.

The compounds provided herein are described with reference to both generic formulas and specific compounds. In addition, the compounds of the present disclosure may exist in a number of different forms or derivatives, all within the scope of the disclosure. These include, for example, pharmaceutically acceptable salts, tautomers, stereoisomers, racemic mixtures, regioisomers, prodrugs, solvated forms, different crystal forms or polymorphs, and active metabolites, etc. In certain embodiments, the compounds of the disclosure may contain bonds with hindered rotation such that two separate rotomers, or atropisomer, may be separated and may have advantageous biological activity. It is intended that all of the possible atropisomes are included with the scope of this disclosure.

As used herein, the term “atropisomer” refers to a stereoisomer resulting from restricted rotation about single bonds where the rotation barrier is high enough to permit isolation of the isomeric species. Typically, rotation about the single bond in the molecule is prevented, or greatly slowed, as a result of steric interactions with other parts of the molecule and the substituents at both ends of the single bond are unsymmetrical.

As used herein, the term “enriched for . . . an atropisomer” or “atropisomerically enriched” means that the compound, i.e., mixture of atropisomers, comprises a greater proportion or percentage of the specified atropisomers of the compound, in relative to the other atropisomers, i.e., greater than 50 mole %, such as greater than 50 mole %, 60 mole %, 70 mole %, 80 mole %, 90 mole %, 95 mole %, 98 mole %, 99 mole %, etc. In certain embodiments, atropisomers other than the specified atropisomer are undetectable. In certain embodiments, the compound may comprise nearly 100 mole % or 100 mole % of the is specified atropisomer of the compound. In certain embodiments, the compound is substantially atropisomerically pure. As used herein, the term “substantially pure” means that the compound, i.e., mixture of atropisomers, comprises at least 90 mole %, preferably at least 95 mole %, more preferably at least 98 mole %, and even more preferably at least 99 mole % of one atropisomer. The term “substantially free” means that the compound comprises less than 10 mole %, preferably less than 5 mole %, more preferably less than 2 mole %, and even more preferably less than 1 mole % of one atropisomer.

As used herein, the term “pharmaceutically acceptable salt”, unless otherwise stated, includes salts that retain the biological effectiveness of the free acid/base form of the specified compound and that are not biologically or otherwise undesirable. Contemplated pharmaceutically acceptable salt forms include, but are not limited to, mono, bis, tris, tetrakis, and so on. Pharmaceutically acceptable salts are non-toxic in the amounts and concentrations at which they are administered. The preparation of such salts can facilitate the pharmacological use by altering the physical characteristics of a compound without preventing it from exerting its physiological effect. Useful alterations in physical properties may include, for example, increasing the solubility to facilitate administering higher concentrations of the drug.

Pharmaceutically acceptable salts of the compounds of Formula (I) or (II) include acid addition and base salts. Suitable acid addition salts can be formed from acids which form non-toxic salts. Non-limiting examples may include the acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, 1,5-naphthalenedisulfonic acid and xinafoate salts. Suitable base salts are formed from bases which form non-toxic salts. Non-limiting examples may include the aluminium, arginine, benzathine, calcium, choline, diethylamine, bis(2-hydroxyethyl)amine (diolamine), glycine, lysine, magnesium, meglumine, 2-aminoethanol (olamine), potassium, sodium, 2-Amino-2-(hydroxymethyl)propane-1,3-diol (tris or tromethamine) and zinc salts. Hemisalts of acids and bases may also be formed, for example, hemisulfate and hemicalcium salts. For a review on suitable salts, see, Stahl and Wermuth, Handbook of Pharmaceutical Salts: Properties, Selection, and Use (Wiley-VCH, 2002).

Pharmaceutically acceptable salts of the compound of Formula (I) or (II) may be prepared by one or more of three methods: (i) by reacting the compound of Formula (I) or (II) with the desired acid or base; (ii) by removing an acid- or base-labile protecting group from a suitable precursor of the compound of Formula (I) or (II) or by ring-opening a suitable cyclic precursor, for example, a lactone or lactam, using the desired acid or base; or (iii) by converting one salt of the compound of Formula (I) or (II) to another by a reaction with an appropriate acid or base or by means of a suitable ion exchange column. The three reactions may be typically carried out in solution. The resulting salt may precipitate out and be collected by filtration or may be recovered by evaporation of the solvent. The degree of ionization in the resulting salt may vary from completely ionized to almost non-ionized.

The compound of Formula (I) or (II) and pharmaceutically acceptable salts thereof may exist in unsolvated and solvated forms. As used herein, the term “solvate” refers to a molecular complex comprising the compound of Formula (I) or (II), or a pharmaceutically acceptable salt thereof, and one or more pharmaceutically acceptable solvent molecules. For example, the term “hydrate” is employed when said solvent is water.

The compounds of Formula (I) or (II) may have one or more chiral (asymmetric) centers. The present disclosure encompasses all stereoisomeric forms of the compounds of Formula (I) or (II). Centers of asymmetry that are present in the compounds of Formula (I) or (II) can all independently of one another have (R) or (S) configuration. When bonds to a chiral carbon are depicted as straight lines in the structural formulas of the present disclosure, or when a compound name is recited without an (R) or (S) chiral designation for a chiral carbon, it is understood that both the (R) and (S) configurations of each such chiral carbon and hence each enantiomer or diastereomer and mixtures thereof are embraced within the formula or by the name. The production of specific stereoisomers or mixtures thereof may be identified in the Examples where such stereoisomers or mixtures were obtained, but this in no way limits the inclusion of all stereoisomers and mixtures thereof from being within the scope of the disclosure.

The present disclosure includes all possible enantiomers and diastereomers and mixtures of two or more stereoisomers, for example mixtures of enantiomers and/or diastereomers, in all ratios. Thus, enantiomers are a subject of the present disclosure in enantiomerically pure form, both as levorotatory and as dextrorotatory antipodes, in the form of racemates and in the form of mixtures of the two enantiomers in all ratios. In the case of a cis/trans isomerism, the present disclosure includes both the cis form and the trans form as well as mixtures of these forms in all ratios. The preparation of individual stereoisomers can be carried out, if desired, by separation of a mixture by customary methods, for example by chromatography or crystallization, by use of stereochemically uniform starting materials for the synthesis or by stereoselective synthesis. Optionally, a derivatization can be carried out before separation of stereoisomers. The separation of a mixture of stereoisomers can be carried out in an intermediate step during the synthesis of a compound of Formula (I) or (II), or it can be done on a final racemic product. Absolute stereochemistry may be determined by X-ray crystallography of crystalline products or crystalline intermediates which are derivatized, if necessary, with a reagent containing a stereogenic center of known configuration. Alternatively, absolute stereochemistry may be determined by Vibrational Circular Dichroism (VCD) spectroscopy analysis.

Unless otherwise stated, the structures depicted herein are also meant to include the compounds that differ only in the presence of one or more isotopically enriched atoms, in other words, the compounds wherein one or more atoms are replaced by atoms having the same atomic number, but an atomic mass or mass number different from the atomic mass or mass number which predominates in nature. Such compounds are referred to as a “isotopic variant”. The present disclosure is intended to include all pharmaceutically acceptable isotopic variants of the compounds of Formula (I) or (II). Examples of isotopes suitable for inclusion in the compounds of the present disclosure include, but not limited to, isotopes of hydrogen, such as ²H and ³H; carbon, such as ¹¹C, ¹³C and ¹⁴C; chlorine, such as ³⁶Cl; fluorine, such as ¹⁸F; iodine, such as ¹²³I and ¹²⁵I; nitrogen, such as ¹³N and ¹⁵N; oxygen, such as ¹⁵O, ¹⁷O and ¹⁸O; phosphorus, such as ³²P; and sulfur, such as ³⁵S. Certain isotopic variants of the compounds of Formula (I) or (II), for example those incorporating a radioactive isotope, may be useful in drug and/or substrate tissue distribution studies. Particularly, compounds having the depicted structures that differ only in the replacement with heavier isotopes, such as the replacement of hydrogen by deuterium (²H), can afford certain therapeutic advantages, for example, resulting from greater metabolic stability, increased in vivo half-life, or reduced dosage requirements and, hence, may be utilized in some particular circumstances. Isotopic variants of compounds of Formula (I) or (II) can generally be prepared by conventional techniques known to one skilled in the art or by processes analogous to those described in the accompanying examples and synthesis using an appropriate isotopically-labeled reagent in place of the non-labeled reagent previously employed. In certain embodiments, isotopic variants of compounds of the present disclosure are deuterated variants.

Pharmaceutically acceptable solvates in accordance with the present disclosure may include those wherein the solvent of crystallization may be isotopically substituted, e.g., D₂O, d₆-acetone, d₆-DMSO.

One way of carrying out the present disclosure is to administer a compound of Formula (I) or (II) in the form of a prodrug. Thus, certain derivatives of a compound of Formula (I) or (II) which may have little or no pharmacological activity themselves can, when administered into or onto the body, be converted into a compound of Formula (I) or (II) having the desired activity, for example by hydrolytic cleavage, particularly hydrolytic cleavage promoted by an esterase or peptidase enzyme. Such derivatives are referred to as “prodrugs”. Further information on the use of prodrugs may be found in, e.g., T. Higuchi and W. Stella, “Pro-drugs as Novel Delivery Systems”, Vol. 14, ACS Symposium Series, and E. B. Roche (Ed.), “Bioreversible Carriers in Drug Design”, Pergamon Press, 1987, American Pharmaceutical Association. Reference can also be made to Nature Reviews/Drug Discovery, 2008, 7, 355, and Current Opinion in Drug Discovery and Development, 2007, 10, 550.

Prodrugs in accordance with the present disclosure can, for example, be produced by replacing appropriate functionalities present in the compounds of Formula (I) or (II) with certain moieties known to those skilled in the art as “pro-moieties” as described, for example, in H. Bundgaard, “Design of Prodrugs”, Elsevier, 1985, and Y. M. Choi-Sledeski and C. G. Wermuth, “Designing Prodrugs and Bioprecursors”, Practice of Medicinal Chemistry, 4^{th} Edition, Chapter 28, 657-696, Elsevier, 2015. Thus, a prodrug in accordance with the present disclosure may include, but not limited to, (a) an ester or amide derivative of a carboxylic acid in a compound of Formula (I) or (II), if any; (b) an amide, imine, carbamate or amine derivative of an amino group in a compound of Formula (I) or (II); (c) an oxime or imine derivative of a carbonyl group in a compound of Formula (I) or (II), if any; or (d) a methyl, primary alcohol or aldehyde group that can be metabolically oxidized to a carboxylic acid in a compound of Formula (I) or (II), if any.

References to compounds of Formula (I) or (II) are taken to include the compounds themselves and prodrugs thereof. The present disclosure includes such compounds of Formula (I) or (II) as well as pharmaceutically acceptable salts of such compounds said compounds and salts.

### Administration and Dosing

The compounds of the present disclosure may be administered in an amount effective to treat the diseases or conditions as described herein. The compounds of the present disclosure can be administered as compound per se, or alternatively, as a pharmaceutically acceptable salt. For administration and dosing purposes, the compound of the present disclosure per se or pharmaceutically acceptable salt, stereoisomer, or isotopic variant thereof will simply be referred to as the compounds of the invention.

The compounds of the disclosure may be administered by any suitable route in the form of a pharmaceutical composition adapted to such a route, and in a dose effective for the treatment intended. The compounds of the disclosure may be administered in various routes, including, e.g., orally, rectally, vaginally, parenterally, topically, etc.

As used herein, the terms “administration” and “administer” refer to absorbing, ingesting, injecting, inhaling, implanting, or otherwise introducing the compound of the disclosure, or a pharmaceutical composition thereof. The terms “treatment” and “treat” refer to reversing, alleviating, delaying the onset of, or inhibiting the progress of a “pathological condition” (e.g., a disease, disorder, or condition, or one or more signs or symptoms thereof) described herein. In certain embodiments, treatment may be administered after one or more signs or symptoms of a disease or condition have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence. As used herein, the terms “disease”, “disorder”, “condition”, and “pathological condition” are used interchangeably.

Dosage levels for administration can be determined by those skilled in the art by routine experimentation. The dosage regimen for the compounds of the disclosure and/or compositions comprising said compounds is based on a variety of factors, including the type, age, weight, sex, and medical condition of the patient; the severity of the condition; the route of administration; and the activity of the particular compound employed. Thus, the dosage regimen may vary widely. It is not uncommon that the administration of the compounds of the disclosure will be repeated a plurality of times in a day.

In certain embodiments, the compound of the disclosure may be used in combination with one or more of additional therapeutical agents. In certain embodiments, non-limiting examples of the additional therapeutical agent may include an anti-cancer agent. In certain embodiments, non-limiting examples of the additional therapeutical agents may include an additional PKMYT1 inhibitor. In certain embodiments, one or more additional therapeutic agent(s) may be selected from the group consisting of: a cytotoxic agent; an antimetabolite; an alkylating agent; an anthracycline; an antibiotic; an anti-mitotic agent; a hormone therapy; a signal transduction inhibitor; a gene expression modulator; an apoptosis inducer; an angiogenesis inhibitor; an immunotherapy agent; a DNA damage repair inhibitor; or a combination thereof.

The additional therapeutical agent can be administered before, after, or at the same time that the compound of the present disclosure is administered.

### Pharmaceutical Compositions

In some aspect, the present disclosure is directed to a pharmaceutical composition comprising the compound of Formula (I) or (II) or a pharmaceutically acceptable salt, stereoisomer, or isotopic variant thereof as provided herein, and at least one pharmaceutically acceptable carrier or excipient.

As used herein, the term “pharmaceutically acceptable carrier or excipient” refers to a carrier or excipient which is useful for preparing a pharmaceutical composition that is generally safe, non-toxic, and neither biologically nor otherwise undesirable, and includes carrier or excipient that is acceptable for veterinary use as well as human pharmaceutical use. A pharmaceutically acceptable carrier or excipient as used herein includes both one and more than one such carrier or excipient. The particular carrier or excipient used will depend upon the means and purpose for which the compounds of the disclosure is being applied. Suitable carriers and excipients are well known to those skilled in the art and are described in detail in, e.g., Ansel, Howard C, et al., Ansel's Pharmaceutical Dosage Forms and Drug Delivery Systems. Philadelphia: Lippincott, Williams & Wilkins, 2004; Gennaro, Alfonso R., et al., Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more of buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents, and other known additives to provide an elegant presentation of the drug (i.e., the compound or pharmaceutical composition as provided herein) or aid in the manufacturing of the pharmaceutical product (i.e., medicament).

The compositions of the present disclosure may be in a variety of forms. These include, for example, liquid, semi-solid and solid dosage forms, such as liquid solutions (e.g., injectable and infusible solutions), dispersions or suspensions, tablets, pills, powders, liposomes, suppositories, etc. The form depends on the intended mode of administration and therapeutic application.

Pharmaceutical compositions of the present disclosure may be prepared by any of the well-known techniques of pharmacy, such as effective formulation and administration procedures. The above considerations in regard to effective formulations and administration procedures are well known in the art, and are described in standard textbooks. Formulation of pharmaceutical products is discussed in, e.g., Hoover, John E., Remington's Pharmaceutical Sciences, Mack Publishing Co., Easton, Pennsylvania, 1975; Liberman, et al., Eds., Pharmaceutical Dosage Forms, Marcel Decker, New York, N.Y., 1980; and Kibbe, et al., Eds., Handbook of Pharmaceutical Excipients, 3^{rd} Edition, American Pharmaceutical Association, Washington, 1999.

In a further aspect, the present disclosure relates to a kit for treating a PKMYT1-associated disease or condition, which comprises a compound of Formula (I) or (II) or a pharmaceutically acceptable salt, stereoisomer, or isotopic variant thereof as provided herein, a container, and optionally a package insert or label indicating treatment of said disease or condition.

### Methods of Treatment

In a further aspect, the present disclosure is directed to a method of treating a PKMYT1-associated disease or condition in a subject in need thereof, which comprises administering to the subject a therapeutically effective amount of the compound of Formula (I) or (II) or a pharmaceutically acceptable salt, stereoisomer, or isotopic variant thereof as provided herein, owning to the PKMYT1 inhibitory activity of the compound of the present disclosure. In certain embodiments, the compounds of the present disclosure are selective PKMYT1 inhibitors.

As used herein, the term “subject in need thereof” is a subject having a PKMYT1-associated disease or condition, or a subject having an increased risk of developing PKMYT1-associated disease or condition relative to the population at large. In certain embodiments, the subject is a warm-blooded animal. In certain embodiments, the warm-blooded animal is a mammal. In certain embodiments, the warm-blooded animal is a human.

PKMYT1 has been implicated as a potentially important cancer target as it is essential in many cancer cells. Overexpression of PKMYT1 has been observed in various cancers including, for example, hepatocellular carcinoma as well as clear-cell renal-cell carcinoma. PKMyt1 downregulation has a minor role in unperturbed cells but has a more prominent role in cells exposed to DNA damage. Additionally, cells that exhibit high levels of replication stress in addition to defective G1 checkpoint regulation may be particularly sensitive to loss of PKMYT1 function, as these cells will be prone to entering mitosis prematurely with compromised genomic material leading to mitotic catastrophe. Inhibitors of PKMYT1, a regulator of G2-M transition, has been found to be particularly useful in the treatment of tumors (e.g., cancers) harboring CCNE1-amplification or FBXW7 loss-of-function mutations using a synthetic lethal therapeutic strategy. Cancers harboring CCNE1-amplification may include, e.g., uterine cancer, ovarian cancer, breast cancer, gastric cancer, esophageal cancer, lung cancer, and endometrial cancer, etc. Cancers harboring FBXW7 loss-of-function may include, e.g., uterine cancer, colorectal cancer, breast cancer, lung cancer, and esophageal cancer, etc. Moreover, PKMYT1 has been implicated in non-small-cell lung cancer, hepatocellular carcinoma, glioblastoma, neuroblastoma, etc.

In certain embodiments, the PKMYT1-associated disease or condition is tumor, and particularly, advanced solid tumor. In certain embodiments, the PKMYT1-associated disease or condition is selected from the group consisting of uterine cancer, ovarian cancer, breast cancer, gastric cancer, esophageal cancer, lung cancer (e.g., non-small-cell lung cancer), endometrial cancer, colorectal cancer, hepatocellular carcinoma, glioblastoma, neuroblastoma, etc.

The method of treating a PKMYT1-associated disease or condition as described herein may be used as a monotherapy. As used herein, the term “monotherapy” refers to the administration of a single active or therapeutic compound to a subject in need thereof. In certain embodiments, monotherapy will involve administration of a therapeutically effective amount of one of the compounds of the present disclosure or a pharmaceutically acceptable salt, stereoisomer, or isotopic variant thereof, to a subject in need of such treatment.

Depending upon the particular disease or condition to be treated, the method of treating a PKMYT1-associated disease or condition described herein may involve, in addition to administration of the compound of Formula (I) or (II), combination therapy of one or more additional therapeutic agent(s), for example, a second therapeutic agent which is an anti-cancer agent. In certain embodiments, non-limiting examples of the additional therapeutical agents may include an additional PKMYT1 inhibitor. In certain embodiments, one or more additional therapeutic agent(s) may be selected from the group consisting of: a cytotoxic agent; an antimetabolite; an alkylating agent; an anthracycline; an antibiotic; an anti-mitotic agent; a hormone therapy; a signal transduction inhibitor; a gene expression modulator; an apoptosis inducer; an angiogenesis inhibitor; an immunotherapy agent; a DNA damage repair inhibitor; or a combination thereof.

The cytotoxic agent may be, e.g., actinomycin-D, alemtuzumab, alitretinoin, allopurinol, altretamine, amifostine, amphotericin, amsacrine, arsenic trioxide, asparaginase, azacitidine, azathioprine, Bacille Calmette-Guérin (BCG), bendamustine, bexarotene, bevacizumab, bleomycin, bortezomib, busulphan, capecitabine, carboplatin, carfilzomib, carmustine, cetuximab, cisplatin, chlorambucil, cladribine, clofarabine, colchicine, crisantaspase, cyclophosphamide, cyclosporine, cytarabine, cytochalasin B, dacarbazine, dactinomycin, darbepoetin alfa, dasatinib, daunorubicin, 1-dehydrotestosterone, denileukin, dexamethasone, dexrazoxane, dihydroxy anthracin dione, disulfiram, docetaxel, doxorubicin, emetine, epirubicin, erlotinib, epigallocatechin gallate, epoetin alfa, estramustine, ethidium bromide, etoposide, everolimus, filgrastim, finasunate, floxuridine, fludarabine, flurouracil (5-FU), fulvestrant, ganciclovir, geldanamycin, gemcitabine, glucocorticoids, gramicidin D, histrelin acetate, hydroxyurea, ibritumomab, idarubicin, ifosfamide, imatinib, irinotecan, interferons, interferon alfa-2a, interferon alfa-2b, ixabepilone, lactate dehydrogenase A (LDH-A), lenalidomide, letrozole, leucovorin, levamisole, lidocaine, lomustine, mechlorethamine, melphalan, 6-mercaptopurine, mesna, methotrexate, methoxsalen, metoprine, metronidazole, mithramycin, mitomycin-C, mitoxantrone, nandrolone, nelarabine, nilotinib, nofetumomab, oprelvekin, oxaliplatin, paclitaxel, pemetrexed, pentostatin, palifermin, pamidronate, pegademase, pegaspargase, pegfilgrastim, pemetrexed disodium, plicamycin, porfimer sodium, procaine, procarbazine, propranolol, puromycin, quinacrine, radicicol, radioactive isotopes, raltitrexed, rapamycin, rasburicase, salinosporamide A, sargramostim, sunitinib, temozolomide, teniposide, tetracaine, 6-thioguanine, thiotepa, topotecan, toremifene, trastuzumab, treosulfan, tretinoin, valrubicin, vinblastine, vincristine, vindesine, vinorelbine, zoledronate, or a combination thereof.

The antimetabolite may be, e.g., methotrexate, 6-mercaptopurine, 6-thioguanine, cytarabine, 5-fluorouracil decarbazine, cladribine, pemetrexed, gemcitabine, capecitabine, hydroxyurea, mercaptopurine, fludarabine, pralatrexate, clofarabine, cytarabine, decitabine, floxuridine, nelarabine, trimetrexate, thioguanine, pentostatin, or a combination thereof.

The alkylating agent may be, e.g., mechlorethamine, thiotepa, chlorambucil, melphalan, carmustine (BSNU), lomustine (CCNU), cyclothosphamide, busulfan, dibromomannitol, streptozotocin, mitomycin C, cis-dichlorodiamine platinum (II) (DDP) cisplatin, altretamine, cyclophosphamide, ifosfamide, hexamethylmelamine, altretamine, procarbazine, dacarbazine, temozolomide, streptozocin, carboplatin, cisplatin, oxaliplatin, uramustine, bendamustine, trabectedin, semustine, or a combination thereof.

The anthracycline may be, e.g., daunorubicin, doxorubicin, aclarubicin, aldoxorubicin, amrubicin, ansamycin, carubicin, epirubicin, idarubicin, mitoxantrone, valrubicin, or a combination thereof.

The antibiotic may be, e.g., dactinomycin, bleomycin, mithramycin, anthramycin (AMC), ampicillin, bacampicillin, carbenicillin, cloxacillin, dicloxacillin, flucloxacillin, mezlocillin, nafcillin, oxacillin, piperacillin, pivampicillin, pivmecillinam, ticarcillin, aztreonam, imipenem, doripenem, ertapenem, meropenem, cephalosporins, clarithromycin, dirithromycin, roxithromycin, telithromycin, lincomycin, pristinamycin, quinupristin, amikacin, gentamicin, kanamycin, neomycin, netilmicin, paromomycin, tobramycin, streptomycin, sulfamethizole, sulfamethoxazole, sulfisoxazole, demeclocycline, minocycline, oxytetracycline, tetracycline, penicillin, amoxicillin, cephalexin, erythromycin, clarithromycin, azithromycin, ciprofloxacin, levofloxacin, ofloxacin, doxycycline, clindamycin, metronidazole, tigecycline, chloramphenicol, metronidazole, tinidazole, nitrofurantoin, vancomycin, teicoplanin, telavancin, linezolid, cycloserine, rifamycins, polymyxin B, bacitracin, viomycin, capreomycin, quinolones, daunorubicin, doxorubicin, 4′-deoxydoxorubicin, epirubicin, idarubicin, plicamycin, mitomycin-c, mitoxantrone, or a combination thereof.

The anti-mitotic agent may be, e.g., vincristine, vinblastine, vinorelbine, docetaxel, estramustine, ixabepilone, paclitaxel, maytansinoid, a dolastatin, a cryptophycin, or a combination thereof.

The signal transduction inhibitor may be, e.g., imatinib, trastuzumab, erlotinib, sorafenib, sunitinib, temsirolimus, vemurafenib, lapatinib, bortezomib, cetuximab, panitumumab, matuzumab, gefitinib, STI 571, rapamycin, flavopiridol, imatinib mesylate, vatalanib, semaxanib, motesanib, axitinib, afatinib, bosutinib, crizotinib, cabozantinib, dasatinib, entrectinib, pazopanib, lapatinib, vandetanib, or a combination thereof.

The gene expression modulator may be, e.g., a siRNA, a shRNA, an antisense oligonucleotide, an HDAC inhibitor, or a combination thereof. An HDAC inhibitor may be, e.g., trichostatin A, trapoxin B, valproic acid, vorinostat, belinostat, LAQ824, panobinostat, entinostat, tacedinaline, mocetinostat, givinostat, resminostat, abexinostat, quisinostat, rocilinostat, pracinostat, CHR-3996, butyric acid, phenylbutyric acid, 4SC202, romidepsin, sirtinol, cambinol, EX-527, nicotinamide, or a combination thereof. An antisense oligonucleotide may be, e.g., custirsen, apatorsen, AZD9150, trabadersen, EZN-2968, LErafAON-ETU, or a combination thereof. An siRNA may be, e.g., ALN-VSP, CALAA-01, Atu-027, SPC2996, or a combination thereof.

The hormone therapy may be, e.g., a luteinizing hormone-releasing hormone (LHRH) antagonist. The hormone therapy may be, e.g., firmagon, leuproline, goserelin, buserelin, flutamide, bicalutamide, ketoconazole, aminoglutethimide, prednisone, hydroxyl-progesterone caproate, medroxy-progesterone acetate, megestrol acetate, diethylstil-bestrol, ethinyl estradiol, tamoxifen, testosterone propionate, fluoxymesterone, flutamide, raloxifene, droloxifene, iodoxyfene, 4-hydroxytamoxifen, trioxifene, keoxifene, LY117018, onapristone, toremifene citrate, megestrol acetate, exemestane, fadrozole, vorozole, letrozole, anastrozole, nilutamide, tripterelin, histrelin, abiraterone, medroxyprogesterone acetate, diethylstilbestrol, premarin, fluoxymesterone, tretinoin, fenretinide, troxacitabine, or a combination thereof.

The apoptosis inducer may be, e.g., a recombinant human TNF-related apoptosis-inducing ligand (TRAIL), camptothecin, bortezomib, etoposide, tamoxifen, or a combination thereof.

The angiogenesis inhibitor may be, e.g., sorafenib, sunitinib, pazopanib, everolimus, or a combination thereof.

The immunotherapy agent may be, e.g., a monoclonal antibody, cancer vaccine (e.g., a dendritic cell (DC) vaccine), oncolytic virus, cytokine, adoptive T cell therapy, Bacille Calmette-Guérin (BCG), GM-CSF, thalidomide, lenalidomide, pomalidomide, imiquimod, or a combination thereof. The monoclonal antibody may be, e.g., anti-CTLA4, anti-PD1, anti-PD-L1, anti-LAG3, anti-KIR, or a combination thereof. The monoclonal antibody may be, e.g., alemtuzumab, trastuzumab, ibritumomab tiuxetan, brentuximab vedotin, trastuzumab, ado-trastuzumab emtansine, blinatumomab, bevacizumab, cetuximab, pertuzumab, panitumumab, ramucirumab, obinutuzumab, ofatumumab, rituximab, tositumomab, gemtuzumab ozogamicin, tositumomab, or a combination thereof. The cancer vaccine may be, e.g., Sipuleucel-T, BioVaxlD, NeuVax, DCVax, SuVaxM, CIMAvax®, Provenge®, hsp110 chaperone complex vaccine, CDX-1401, MIS416, CDX-110, GVAX Pancreas, HyperAcute™ Pancreas, GTOP-99 (MyVax®), or Imprime PGG®. The oncolytic virus may be, e.g., talimogene laherparepvec. The cytokine may be, e.g., IL-2, IFNα, or a combination thereof. The adoptive T cell therapy may be, e.g., tisagenlecleucel, axicabtagene ciloleucel, or a combination thereof.

The DNA damage repair inhibitor may be, e.g., a PARP inhibitor, a cell checkpoint kinase inhibitor, or a combination thereof. The PARP inhibitor may be, e.g., olaparib, rucaparib, veliparib (ABT-888), niraparib (ZL-2306), iniparib (BSI-201), talazoparib (BMN 673), 2X-121, CEP-9722, KU-0059436 (AZD2281), PF-01367338, or a combination thereof. The cell checkpoint kinase inhibitor may be, e.g., MK-1775 or AZD1775, AZD7762, LY2606368, PF-0477736, AZD0156, GDC-0575, ARRY-575, CCT245737, PNT-737, or a combination thereof.

As used herein, the term “combination therapy” refers to the administration of a combination of multiple active therapeutic agents. In certain embodiments, the compound of the present disclosure or a pharmaceutically acceptable salt, stereoisomer, or isotopic variant thereof may be administered simultaneously, separately or sequentially to treatment with the one or more additional therapeutic agent(s). For example, the additional therapeutic agent(s) may be administered separately from the compound of the present disclosure, as part of a multiple dosage regimen. Alternatively, the additional therapeutic agent(s) may be part of a single dosage form, mixed with the compound of the present disclosure in a single composition.

In a further aspect, the present disclosure is directed to the compound of Formula (I) or (II) or a pharmaceutically acceptable salt, stereoisomer, or isotopic variant thereof as provided herein for use in the treatment of a PKMYT1-associated disease or condition.

In a further aspect, the present disclosure is directed to use of the compound of Formula (I) or (II) or a pharmaceutically acceptable salt, stereoisomer, or isotopic variant thereof as provided herein in the manufacture of a medicament for treating a PKMYT1-associated disease or condition.

### Synthesis

The compounds of the present disclosure may be prepared by the general and specific methods described below, using the common general knowledge of those skilled in the art of synthetic organic chemistry. Such common general knowledge can be found in standard reference books, e.g., Barton and Ollis (Ed.), Comprehensive Organic Chemistry, Elsevier; Richard Larock, Comprehensive Organic Transformations: A Guide to Functional Group Preparations, John Wiley and Sons; and Compendium of Organic Synthetic Methods, Vol. I-XII, Wiley-Interscience. The starting materials used herein are commercially available or may be prepared by routine methods known in the art.

The Schemes described hereinafter are intended to provide a general description of the methodology employed in the preparation of the compounds of the present disclosure. Some of the compounds of the present disclosure may contain single or multiple chiral centers with the stereochemical designation (R) or (S). It will be apparent to those skilled in the art that all of the synthetic transformations can be conducted in a similar manner no whether the materials are enantioenriched or racemic. Moreover, the resolution to the desired optically active material may take place at any desired point in the procedure using well known methods such as those described herein and in the chemistry literature.

### EXAMPLES

In order that the disclosure may be more fully understood, the following examples are set forth. The examples described herein are offered to illustrate the compounds, methods and compositions provided herein and are not to be construed in any way as limiting the scope of the disclosure.

During synthetic procedures, it may be necessary and/or desirable to protect sensitive or reactive groups on any of the molecules concerned. This may be achieved by means of conventional protecting groups, such as those described in T. W. Greene and P. G. M. Wutts, Protective Groups in Organic Synthesis, 4^{th} Edition, John Wiley and Sons. The protective groups are optionally removed at a convenient subsequent stage using methods well known in the art.

The compounds of the present disclosure can be readily prepared according to the following reaction schemes and examples, or modifications thereof, using readily available starting materials, reagents, and conventional synthesis procedures. In these reactions, it is also possible to make use of variants which are themselves known to those skilled in the art, but are not mentioned in greater detail. Furthermore, other methods for preparing the compounds of the disclosure will be readily apparent to those skilled in the art in light of the reaction schemes and examples as described herein. Unless otherwise indicated, all variables are as defined above.

In general chemical procedures, all reagents and materials may be purchased from commercial vendors or may be readily prepared by those skilled in the art. A list of abbreviations for reagents used and organic moieties may be found in Table 1, below.

TABLE 1
Abbreviations of reagents or organic moieties

| Abbreviation | Full Name |
|---|---|
| HOAc | acetic acid |
| aq. | Aqueous |
| EA | ethyl acetate |
| DCM | dichloromethane |
| DME | dimethyl ether |
| DMF | dimethylformamide |
| DMSO | dimethyl sulfoxide |
| Me | methyl |
| NCS | N-chlorosuccinimide |
| LCMS | HPLC with mass spectral detection |
| Pd(dtbpf)Cl₂ | [1,1′-Bis(di-tert-butylphosphino)ferrocene]dichloropalladium(II) |
| Pre-HPLC | preparative high performance liquid chromatography |
| Pre-TLC | preparative thin layer chromatography |
| r.t. | room temperature |
| RT | retention time |
| sat. | saturated |
| TBAF | tetrabutyl ammonium fluoride |
| THF | tetrahydrofuran |
| TFA | trifluoroacetic acid |

### Example 1

### Synthesis of Compound 1:

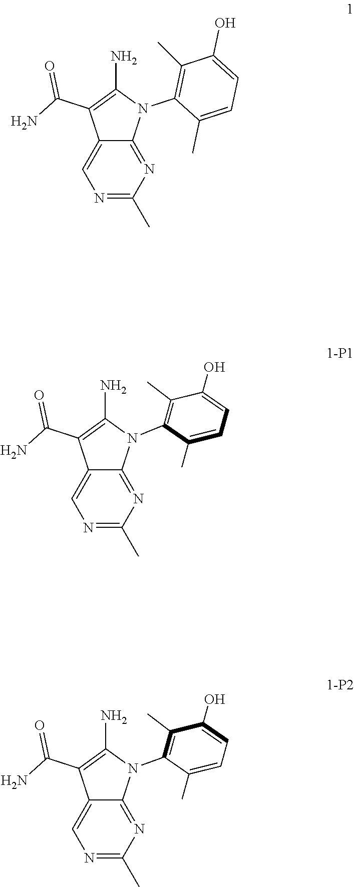

The title compounds were synthesized according to the following synthetic procedures.

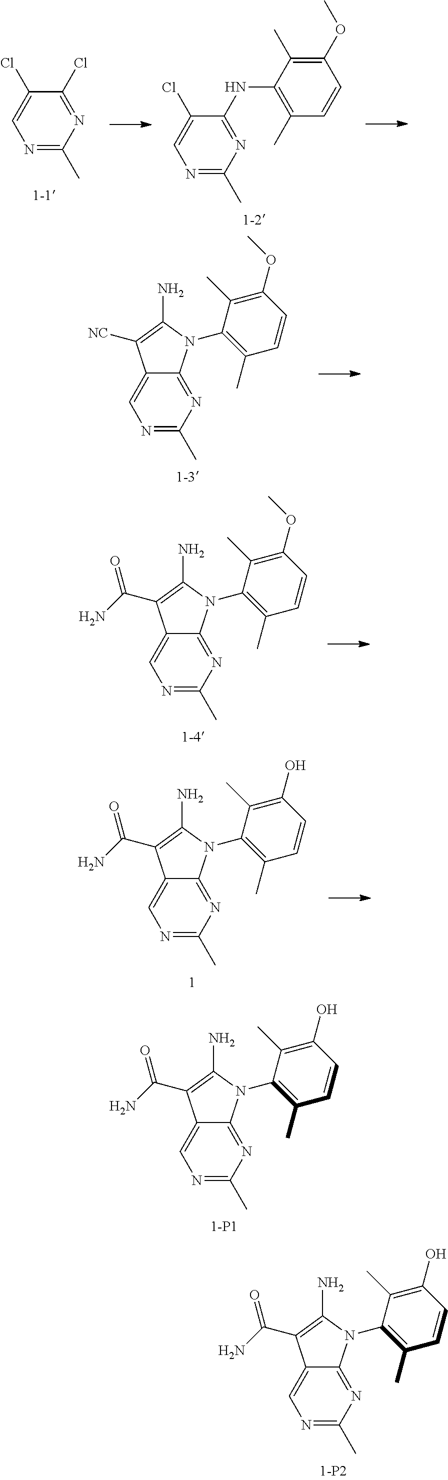

### Preparation of Intermediate 1-2′

To a solution of 4,5-dichloro-2-methylpyrimidine (1.00 g, 6.135 mmol) and 3-methoxy-2,6-dimethylaniline (927.7 mg, 6.135 mmol) in dioxane (20 mL) were added XantPhos Pd G3 (290.9 mg, 0.307 mmol) and Cs₂CO₃ (6.00 g, 18.405 mmol). The mixture was stirred at 110° C. under N₂ for 18 hr. The mixture was filtered. The filtrate was concentrated to dryness. The residue was purified by silica gel column to give 5-chloro-4-[(3-methoxy-2,6-dimethylphenyl)amino]-2-methylpyrimidine (450 mg, 26.41%) as a yellow solid.

LCMS: 278.2 [M+H]⁺.

### Preparation of Intermediate 1-3′

To a solution of propanedinitrile (199.8 mg, 3.024 mmol) in DME (1 mL) was added NaH (126.7 mg, 3.168 mmol) at 0° C. The mixture was stirred for 0.5 hr. Then Brettphos Pd G3 (130.6 mg, 0.144 mmol) and 5-chloro-4-[(3-methoxy-2,6-dimethylphenyl)amino]-2-methyl pyrimidine (400 mg, 1.440 mmol) were added. The mixture was stirred at 100° C. for 3 hr. The mixture was filtered. The filtrate was concentrated and purified by silica gel column to give 6-amino-7-(3-methoxy-2,6-dimethylphenyl)-2-methylpyrrolo[2,3-d]pyrimidine-5-carbonitrile as a yellow solid.

LCMS: 308.2 [M+H]⁺.

### Preparation of Intermediate 1-4′

A mixture of 6-amino-7-(3-methoxy-2,6-dimethylphenyl)-2-methylpyrrolo[2,3-d]pyrimidine-5-carbonitrile (140 mg, 0.455 mmol) in H₂SO₄ (2 mL) was stirred at 15° C. for 0.5 hr. The mixture was quenched with ice-water (15 mL), basified to pH˜8 with 2M NaOH, and extracted with DCM (20 mL×3). The organic layers were combined, dried over Na₂SO₄, and concentrated to dryness. The residue was purified by silica gel column to give 6-amino-7-(3-methoxy-2,6-dimethylphenyl)-2-methylpyrrolo[2,3-d]pyrimidine-5-carboxamide as a yellow solid.

LCMS: 326.4 [M+H]⁺.

### Preparation of Compound 1

To a solution of 6-amino-7-(3-methoxy-2,6-dimethylphenyl)-2-methylpyrrolo[2,3-d]pyrimidine-5-carboxamide (90 mg, 0.277 mmol) in DCM (3 mL) was added tribromoborane (0.133 mL, 1.383 mmol). The mixture was stirred at 15° C. for 2 hr. The reaction was quenched with MeOH (1 mL). The mixture was concentrated and purified by Pre-HPLC to give 6-amino-7-(3-hydroxy-2,6-dimethylphenyl)-2-methylpyrrolo[2,3-d] pyrimidine-5-carboxamide as a white solid. Chiral resolution of Compound 1 was conducted by Prep-SFC (column: Daicel Chiralcel® AD, 250*25 mm, 10 μm; Mobile phase A: CO₂, Mobile phase B: EtOH (+0.10% 7.0 mol/L Ammonia in EtOH); Flow rate 70 mL/min: Gradient: isocratic 40% B; column temperature 25° C.; Back pressure: 100 bar; wave length: 214 nm; RT₁ (min): 4.150 (Compound 1-P1), RT₂ (min): 4.368 (Compound 1-P2).

LCMS: 312.1 [M+H]⁺;

¹HNMR: (400 MHz, CD₃OD) δ 8.78 (s, 1H), 7.11 (d, J=8.3 Hz, 1H), 6.94 (d, J=8.3 Hz, 1H), 2.56 (s, 3H), 1.85 (s, 3H), 1.80 (s, 3H).

### Example 2

### Synthesis of Compound 2:

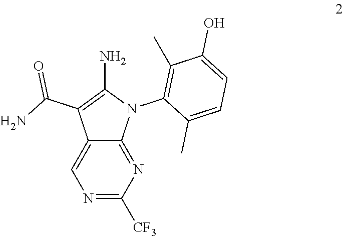 [SMILES: Cc1ccc(O)c(C)c1-n1c(N)c(C(N)=O)c2cnc(C(F)(F)F)nc21]

The title compound was synthesized according to the following synthetic procedures.

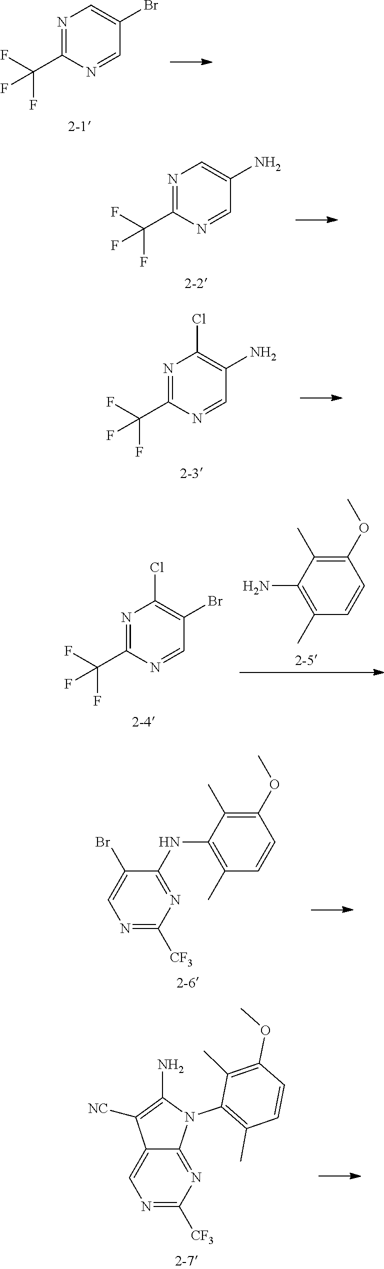

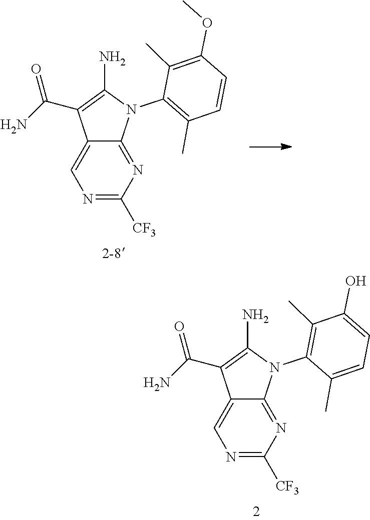 [SMILES: COc1ccc(C)c(-n2c(N)c(C(N)=O)c3cnc(C(F)(F)F)nc32)c1C ; Cc1ccc(O)c(C)c1-n1c(N)c(C(N)=O)c2cnc(C(F)(F)F)nc21]

### Preparation of Intermediate 2-2′

To a solution of Compound 2-1′ (1.5 g, 6.609 mmol) in DMSO (15 mL) were added CuI (0.25 g, 1.322 mmol), L-proline (0.30 g, 2.643 mmol), and K₂CO₃ (1.37 g, 9.913 mmol). The mixture was stirred at room temperature for 5 min. Then ammonium hydroxide (0.382 mL, 9.913 mmol) was added. The mixture was stirred at 85° C. overnight, quenched with water (30 mL), and extracted with EA (50 mL). The organic layer was separated, washed with brine, and concentrated in vacuo. The residue was purified using silica gel column chromatography to afford Intermediate 2-2′ (0.730 g, 67.7%) as white solid.

LCMS: 164.3 [M+H]⁺.

### Preparation of Intermediate 2-3′

To a solution of Intermediate 2-2′ (0.700 g, 4.292 mmol) in DMF (10 mL) was added NCS (0.860 g, 6.438 mmol). The reaction was stirred at room temperature for overnight. The reaction was diluted with EA (50 mL) and water (30 mL). The organic layer was separated, washed with brine, and concentrated in vacuo. The residue was purified using silica gel column chromatography eluting to afford Intermediate 2-3′ (0.60 g, 70.77%) as a yellow solid.

LCMS: 239.1 [M+CH₃CN+H]⁺.

### Preparation of Intermediate 2-4′

To a solution of Intermediate 2-3′ (0.40 g, 2.025 mmol) in CH₃CN (4 mL) were added CuBr (0.436 g, 3.037 mmol) and tert-Butyl nitrite (0.356 g, 3.037 mmol). The mixture was stirred at 60° C. for 3 hr. The reaction was diluted with water (30 mL), and extracted with EA (30 mL). The organic layer was separated, washed with brine, and concentrated in vacuo. The residue was purified using silica gel column chromatography to afford Intermediate 2-4′ (0.200 g, 37.78%) as a colorless oil.

¹HNMR: (400 MHz, DMSO-d₆) δ 9.42 (s, 1H).

### Preparation of Intermediate 2-6′

To a solution of Intermediate 2-4′ (0.200 g, 0.765 mmol) and 3-methoxy-2,6-dimethylaniline (0.139 g, 0.918 mmol) in t-BuOH (2 mL) were added TFA (0.0872 g, 0.765 mmol). The reaction was stirred at 100° C. overnight. The reaction mixture was cooled to room temperature, diluted with EA and water. The organic layer was separated, washed with saturated NaHCO₃ solution, and concentrated in vacuo. The residue was purified using silica gel column chromatography to afford Intermediate 2-6′ (0.100 g, 34.75%) as a colorless oil.

LCMS: 419.2 [M+CH₃CN+H]⁺.

### Preparation of Intermediate 2-7′

To a solution of propanedinitrile (28.1 mg, 0.425 mmol) in DME (1 mL) was added NaH (25.5 mg, 0.638 mmol) at 0° C. The mixture was stirred for 0.5 hr. Then XantPhos Pd G3 (40.3 mg, 0.043 mmol) and Intermediate 2-6′ (80 mg, 0.213 mmol) were added. The mixture was stirred at 110° C. for 1 hr by microwave. The mixture was filtered. The filtrate was concentrated and purified by silica gel column to give Intermediate 2-7′ as a yellow solid.

LCMS: 403.3 [M+CH₃CN+H]⁺.

### Preparation of Intermediate 2-8′

A solution of Intermediate 2-7′ (42 mg, 0.116 mmol) in H₂SO₄ (2 mL) was stirred at room temperature for 30 min. The reaction was diluted with ice-water and basified with 2M NaOH until pH˜8. The mixture was extracted with DCM (40 mL). The organic layer was separated, washed with brine, dried over Na₂SO₄, filtered, and concentrated to afford Intermediate 2-8′ as a yellow solid.

LCMS: 380.3 [M+H]⁺.

### Preparation of Compound 2

To a solution of Intermediate 2-8′ (40 mg, 0.105 mmol) in DCM (2 mL) was added boron tribromide (0.2 mL). The solution was stirred at room temperature for 1 hr. The reaction was quenched with MeOH (1 mL). The mixture was concentrated and purified by Pre-HPLC to afford Compound 2 as a white solid.

LCMS: 366.3 [M+H]⁺;

¹HNMR: (400 MHz, DMSO-d₆) δ 9.68 (s, 1H), 9.12 (s, 1H), 7.50 (s, 2H), 7.13 (s, 1H), 7.11 (s, 2H), 6.98 (d, J=1.2 Hz, 1H), 1.75 (s, 3H), 1.67 (s, 3H).

### Example 3

### Synthesis of Compound 3:

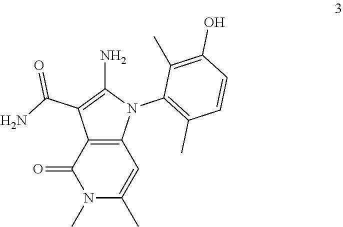 [SMILES: Cc1ccc(O)c(C)c1-n1c(N)c(C(N)=O)c2c(=O)n(C)c(C)cc21]

The title compound was synthesized according to the following synthetic procedures.

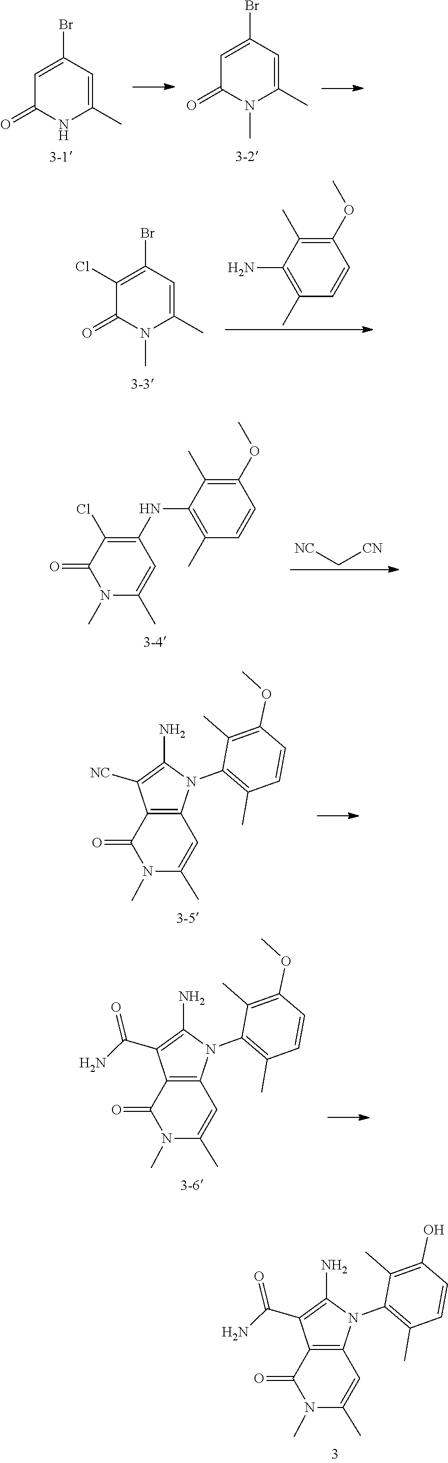

### Preparation of Intermediate 3-2′

To a solution of 4-bromo-6-methyl-1,2-dihydropyridin-2-one (3.70 g, 19.68 mmol) in THF (10 mL) were added TBAF (78.7 mL, 78.72 mmol) and CH₃I (2.45 mL, 39.36 mmol). The mixture was stirred at 60° C. for 16 hr. The mixture was cooled to room temperature, diluted with EA (100 mL), washed with water (100 mL). The organic layer was dried over Na₂SO₄, filtered and concentrated, and purified by silica gel column to give 4-bromo-1,6-dimethyl-1,2-dihydropyridin-2-one (3.50 g, 88.03%) as a yellow solid.

LCMS: 204.0 [M+H]⁺;

¹HNMR: (400 MHz, CDCl₃) δ 6.72 (d, J=4.0 Hz, 1H), 6.23 (s, 1H), 3.48 (s, 3H), 2.34 (s, 3H).

### Preparation of Intermediate 3-3′

To a solution of 4-bromo-1,6-dimethyl-1,2-dihydropyridin-2-one (1000 mg, 4.949 mmol) in DMF (10 mL) was added NCS (726.96 mg, 5.444 mmol). The mixture was stirred at 45° C. for 16 hr. The reaction mixture was diluted with EA (100 mL), which was washed with brine (100 mL×3). The organic layer was dried over Na₂SO₄, filtered and concentrated. The residue was purified by silica gel column to give 4-bromo-3-chloro-1,6-dimethyl-1,2-dihydropyridin-2-one (1.00 g, 85.44%) as a yellow solid.

LCMS: 237.9 [M+H]⁺.

### Preparation of Intermediate 3-4′

To a solution of 4-bromo-3-chloro-1,6-dimethyl-1,2-dihydropyridin-2-one (500 mg, 2.114 mmol) in dioxane (10 mL) were added 3-methoxy-2,6-dimethylaniline (352 mg, 2.326 mmol), XantPhos Pd G3 (240.6 g, 0.254 mmol), and Cs₂CO₃ (2.07 g, 6.343 mmol), and the mixture was stirred at 110° C. for 16 hr and cooled to room temperature. The mixture was diluted with EA (100 mL) and water (30 mL). The organic layer was separated, washed with brine (30 mL), dried over Na₂SO₄, filtered, and concentrated in vacuo. The residue was purified using silica gel column chromatography to afford 3-chloro-4-[(3-methoxy-2,6-dimethylphenyl) amino]-1,6-dimethyl-1,2-dihydropyridin-2-one (230 mg, 35.46%).

LCMS: 307.1 [M+H]⁺.

### Preparation of Intermediate 3-5′

To a solution of propanedinitrile (75.79 mg, 1.147 mmol) in DMF (3 mL) was added NaH (46 mg, 1.147 mmol) at 0° C. The mixture was stirred for 0.5 hr. Then CuI (4.9 mg, 0.026 mmol), methyl[(1S,2S)-2-(methylamino)cyclohexyl]amine (7.42 mg, 0.052 mmol), and 3-chloro-4-[(3-methoxy-2,6-dimethylphenyl)amino]-1,6-dimethyl-1,2-dihydropyridin-2-one (160 mg, 0.522 mmol) were added. The mixture was stirred at 150° C. for 1 hr (microwave). The mixture was quenched with water (10 mL) and extracted with EA (10 mL). The organic layer was washed with brine (10 mL), dried over Na₂SO₄, filtered, and concentrated. The residue was purified by Pre-TLC to give 2-amino-1-(3-methoxy-2,6-dimethylphenyl)-5,6-dimethyl-4-oxopyrrolo[3,2-c]pyridine-3-carbonitrile as a yellow solid.

LCMS: 337.1 [M+H]⁺.

### Preparation of Intermediate 3-6′

A mixture of 2-amino-1-(3-methoxy-2,6-dimethylphenyl)-5,6-dimethyl-4-oxopyrrolo[3,2-c]pyridine-3-carbonitrile (50 mg, 0.149 mmol) in H₂SO₄ (2 mL) was stirred at 15° C. for 0.5 hr. The mixture was quenched with ice-water (15 mL), basified to pH˜8 with 2M NaOH. The mixture was extracted with DCM (20 mL×3). The organic layers were combined dried over Na₂SO₄, and concentrated to dryness. The residue was purified by Pre-TLC to give 2-amino-1-(3-methoxy-2,6-dimethylphenyl)-5,6-dimethyl-4-oxopyrrolo[3,2-c]pyridine-3-carboxamide as a white solid.

LCMS: 355.1 [M+H]⁺.

### Preparation of Compound 3

To a solution of 6-amino-7-(3-methoxy-2,6-dimethylphenyl)-2-methylpyrrolo[2,3-d] pyrimidine-5-carboxamide (90 mg, 0.277 mmol) in DCM (1 mL) was added tribromoborane (0.3 μL, 0.027 mmol). The mixture was stirred at 15° C. for 2 hr. The reaction was quenched with MeOH (1 mL). The mixture was concentrated and purified by Pre-HPLC to give 2-amino-1-(3-hydroxy-2,6-dimethylphenyl)-5,6-dimethyl-4-oxopyrrolo[3,2-c]pyridine-3-carboxamide as a white solid.

LCMS: 341.0 [M+H]⁺;

¹HNMR: (400 MHz, DMSO-d₆) δ 10.33 (s, 1H), 9.63 (s, 1H), 7.09 (t, J=7.8 Hz, 1H), 6.94 (d, J=8.2 Hz, 1H), 6.64 (s, 1H), 6.45 (s, 1H), 6.27 (s, 1H), 5.67 (s, 1H), 3.49 (s, 3H), 2.30 (s, 3H), 1.78 (s, 3H), 1.69 (s, 3H).

### Example 4

### Synthesis of Compound 4:

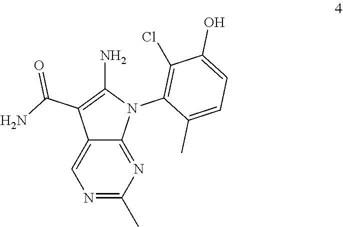 [SMILES: Cc1ncc2c(C(N)=O)c(N)n(-c3c(C)ccc(O)c3Cl)c2n1]

The title compound was synthesized according to the following synthetic procedures.

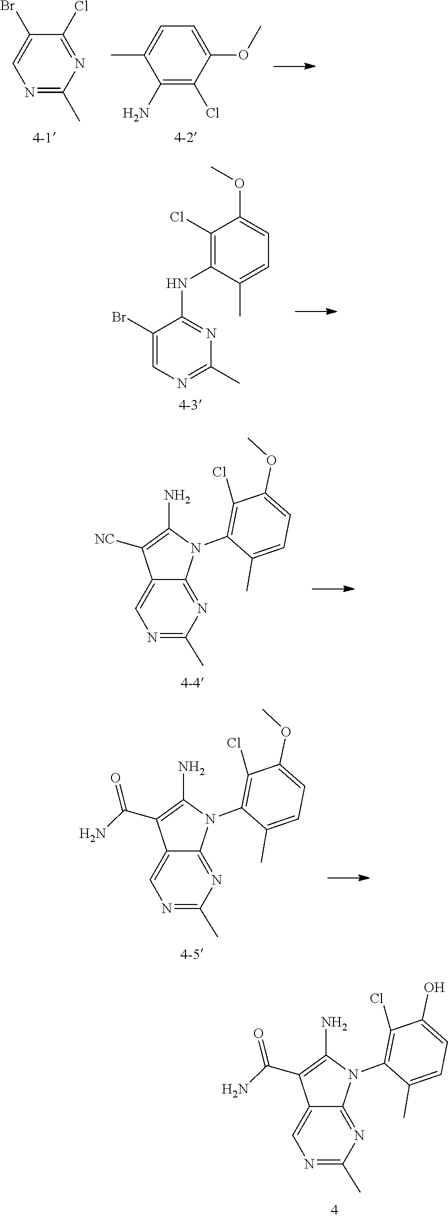

### Preparation of Intermediate 4-3′

To a solution of 5-bromo-4-chloro-2-methylpyrimidine (230 mg, 1.109 mmol) and 2-chloro-3-methoxy-6-methylaniline (209.29 mg, 1.220 mmol) in t-BuOH (20 mL) was added TFA (126.41 mg, 1.109 mmol). The solution was stirred at 110° C. for 16 hr. The reaction mixture was cool to r.t., diluted with EA (30 mL), and washed with brine (30 mL). The organic layer was dried over Na₂SO₄, filtered, concentrated, and purified by silica gel column chromatography to afford 5-bromo-4-[(2-chloro-3-methoxy-6-methylphenyl)amino]-2-methylpyrimidine (60 mg, 15.8%) as a yellow oil.

LCMS: 343.9 [M+H]⁺.

### Preparation of Intermediate 4-4′

To a solution of propanedinitrile (14.46 mg, 0.219 mmol) in DME (1 mL) was added NaH (9.34 mg, 0.233 mmol) at 0° C. The mixture was stirred for 0.5 hr. Then XantPhos Pd G3 (13.84 mg, 0.015 mmol) and 5-bromo-4-[(2-chloro-3-methoxy-6-methylphenyl)amino]-2-methylpyrimidine (50 mg, 0.146 mmol) were added. The mixture was stirred at 110° C. for 1 hr (microwave). The mixture was diluted with EA (30 mL), and washed with brine (30 mL×3). The organic layer was dried over Na₂SO₄, filtered, concentrated, and purified by Pre-TLC to afford 6-amino-7-(2-chloro-3-methoxy-6-methylphenyl)-2-methylpyrrolo[2,3-d]pyrimidine-5-carbonitrile as a yellow solid.

LCMS: 328.1[M+H]⁺.

### Preparation of Intermediate 4-5′

A solution of 6-amino-7-(2-chloro-3-methoxy-6-methylphenyl)-2-methylpyrrolo[2,3-d]pyrimidine-5-carbonitrile (2 mg, 0.006 mmol) in H₂SO₄ (1 mL) was stirred at 25° C. for 1 hr. The mixture was diluted with ice water (10 mL), neutralized with NaOH aq. to pH˜7, extracted with DCM (5 mL×2), washed with brine, dried over Na₂SO₄, filtered, concentrated to afford 6-bromo-2-chloro-3-methoxyaniline as a yellow oil.

LCMS: 346.1 [M+H]⁺.

### Preparation of Compound 4

To a solution of 6-amino-7-(2-chloro-3-methoxy-6-methylphenyl)-2-methylpyrrolo[2,3-d]pyrimidine-5-carboxamide (20 mg, 0.058 mmol) in DCM (2 mL) was added tribromoborane (72.45 mg, 0.289 mmol). The solution was stirred at 25° C. for 1 hr, quenched with MeOH (3 mL), and concentrated. The residue was purified by Pre-HPLC to afford 6-amino-7-(2-chloro-3-hydroxy-6-methylphenyl)-2-methylpyrrolo[2,3-d] pyrimidine-5-carboxamide as a white solid.

LCMS: 332.1 [M+H]⁺;

¹HNMR: (400 MHz, CD₃OD) δ 8.98 (s, 1H), 8.98 (s, 1H), 7.30 (d, J=8.5 Hz, 1H), 7.16 (d, J=8.5 Hz, 1H), 2.74 (s, 3H), 2.74 (s, 3H), 1.99 (s, 3H).

### Example 5

### Synthesis of Compound 5:

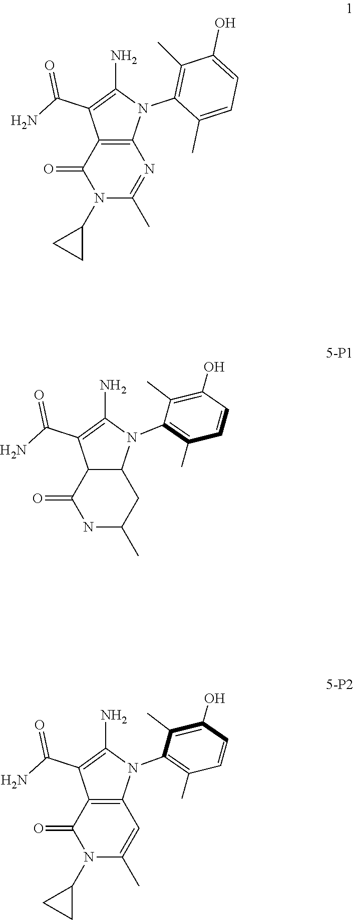

The title compounds were synthesized according to the following synthetic procedures.

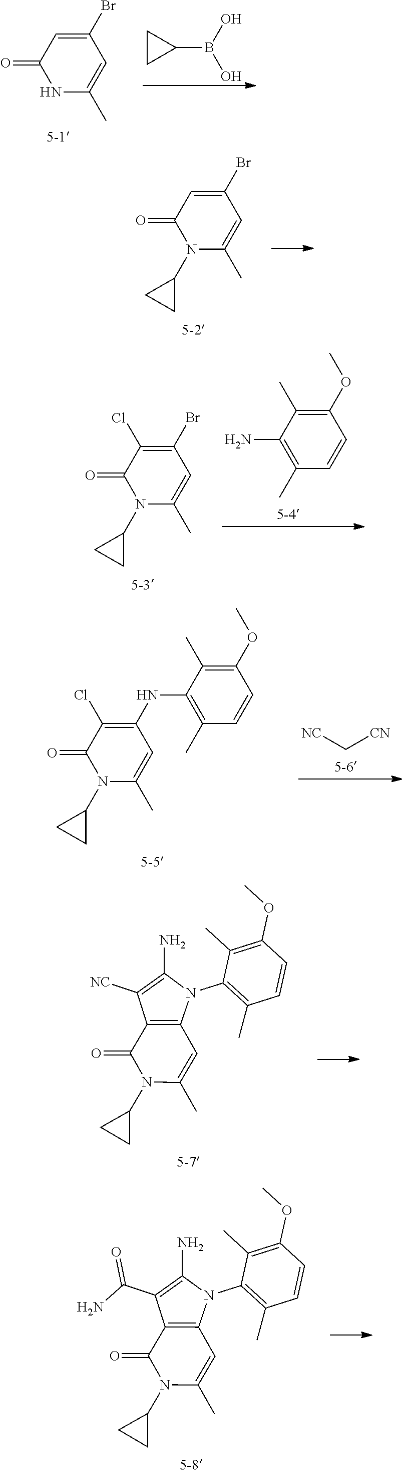

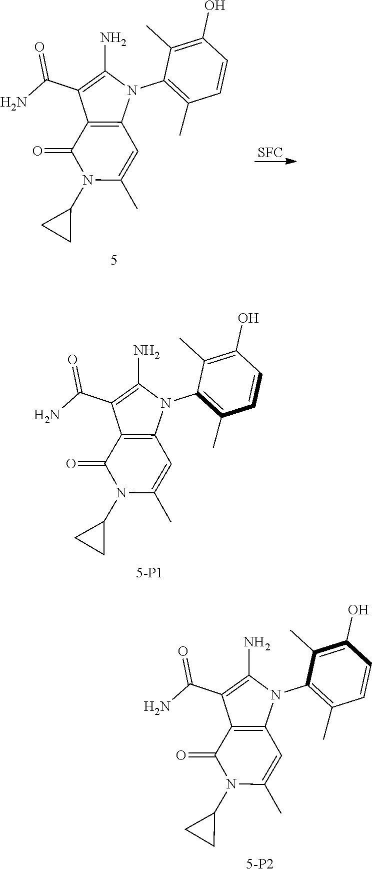

### Preparation of Intermediate 5-2′

To a solution of Compound 5-1′ (2.50 g, 13.296 mmol) in toluene (50 mL) were added cyclopropylboronic acid (2.28 g, 26.593 mmol), cupric bis(acetate) (2.42 g, 13.296 mmol), pyridine (5.35 mL, 66.482 mmol), and potassium bis(trimethylsilyl)azanide (13.3 mL, 13.296 mmol), and the reaction was stirred at 100° C. under O₂ overnight. The mixture was cooled to r.t., diluted with DCM and water. The organic layer was separated, dried, filtered, and concentrated. The residue was purified using silica gel column chromatography to afford Intermediate 5-2′ (1.70 g, 56.05%) as a yellow oil.

LCMS: 230.1 [M+H]⁺.

### Preparation of Intermediate 5-3′

To a solution of Intermediate 5-2′ (1.50 g, 6.576 mmol) in DMF (10 mL) was added NCS (1.05 g, 7.892 mmol). The reaction was stirred at room temperature overnight, diluted with EA and water. The organic layer was separated, washed with brine, and concentrated in vacuo. The residue was purified using silica gel column chromatography to afford Intermediate 5-3′ (0.90 g, 52.13%) as a yellow solid.

LCMS: 264.2 [M+H]⁺.

### Preparation of Intermediate 5-5′

To a solution of Intermediate 5-3′ (850 mg, 3.238 mmol) in dioxane (10 mL) were added 3-methoxy-2,6-dimethylaniline (489.58 mg, 3.238 mmol), Cs₂CO₃ (3.16 g, 9.713 mmol), and XantPhos Pd G3 (460.57 mg, 0.486 mmol). The reaction was stirred at 110° C. for 4 hr. The reaction was diluted with DCM and water. The organic layer was separated, and concentrated in vacuo. The residue was purified using silica gel column chromatography to afford Intermediate 5-5′ (350 mg, 32.48%) as a brown solid.

LCMS: 333.0 [M+H]⁺.

### Preparation of Intermediate 5-7′

To a solution of propanedinitrile (157.20 mg, 2.380 mmol) in DMF (3 mL) at 0° C. was added NaH (57.11 mg, 2.380 mmol) and stirred for 30 min. Intermediate 5-5′ (360 mg, 1.082 mmol), CuI (10.30 mg, 0.054 mmol), and (1S,2S)—N1,N2-dimethylcyclohexane-1,2-diamine (15.39 mg, 0.108 mmol) were added. The reaction was stirred at 140° C. for 1 hr, cooled to r.t., diluted with EA (50 mL), washed with brine (30 mL×3). The organic layer was dried over Na₂SO₄, filtered, and concentrated. The residue was purified using silica gel column chromatography to afford Intermediate 5-7′ as a brown solid.

LCMS: 363.2 [M+H]⁺.

### Preparation of Intermediate 5-8′

A solution of Intermediate 5-7′ (180 mg, 0.497 mmol) in H₂SO₄ (5 mL) was stirred at room temperature for 1.5 hr. The reaction mixture was diluted with DCM and water, neutralized with NaOH (4 M aq.) to pH˜7. The organic phase was dried, and concentrated in vacuo. The residue was purified using silica gel column chromatography to afford Intermediate 5-8′ as a brown solid.

LCMS: 381.4 [M+H]⁺.

### Preparation of Compound 5

To a solution of Intermediate 5-8′ (100 mg, 0.263 mmol) in DCM (3 mL) was added tribromoborane (79.02 mg, 0.315 mmol), and the reaction was stirred at room temperature under N₂ for 30 min. The reaction mixture was diluted with MeOH (10 mL) and water (30 mL), extracted with EA (50 mL×3). The combined organic layer was washed with brine (30 mL), dried over Na₂SO₄, filtered, and concentrated to dryness. The residue was purified by Pre-TLC to afford 2-amino-5-cyclopropyl-1-(3-hydroxy-2,6-dimethylphenyl)-6-methyl-4-oxopyrrolo[3,2-c]pyridine-3-carboxamide as a yellow solid. The enantiomers of Compound 5 were purified by chiral preparative HPLC to afford Compound 5-P1 and Compound 5-P2.

LCMS: 367.3 [M+H]⁺;

¹HNMR: (400 MHz, DMSO-d₆) δ 10.26 (s, 1H), 9.63 (s, 1H), 7.09 (d, J=7.9 Hz, 1H), 6.93 (d, J=8.2 Hz, 1H), 6.63 (s, 1H), 6.42 (s, 2H), 5.61 (s, 1H), 2.95-2.79 (m, 1H), 2.38 (s, 3H), 1.78 (s, 3H), 1.69 (s, 3H), 1.14 (d, J=5.7 Hz, 2H), 0.83 (s, 2H).

### Example 6

### Synthesis of Compound 6:

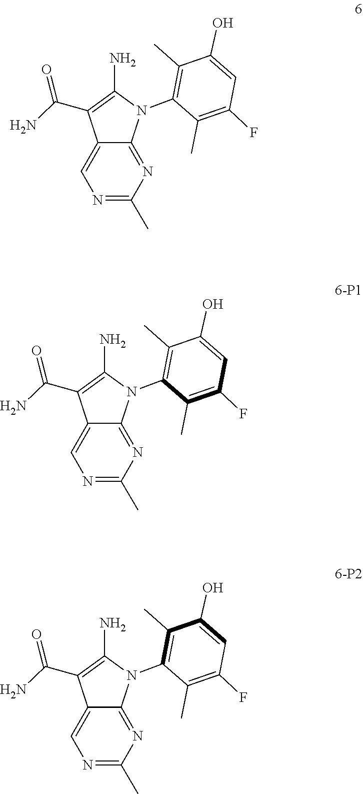

The title compounds were synthesized according to the following synthetic procedures.

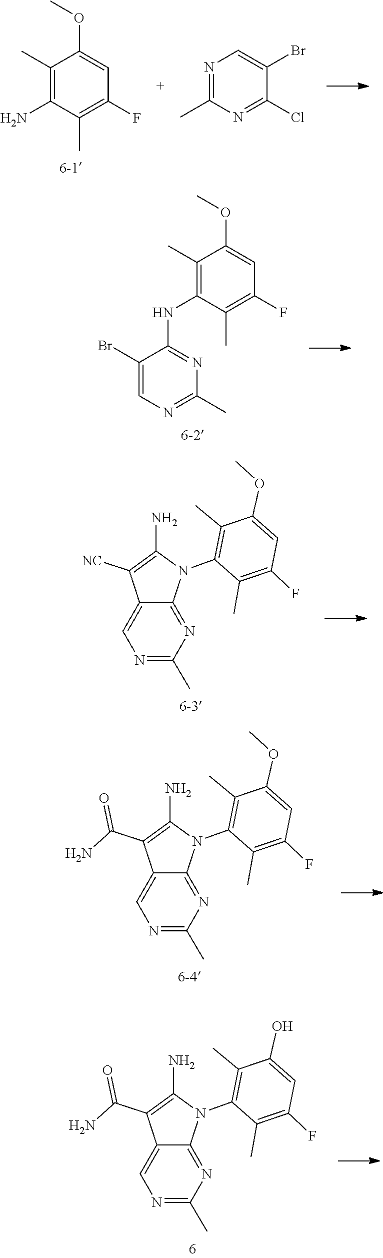

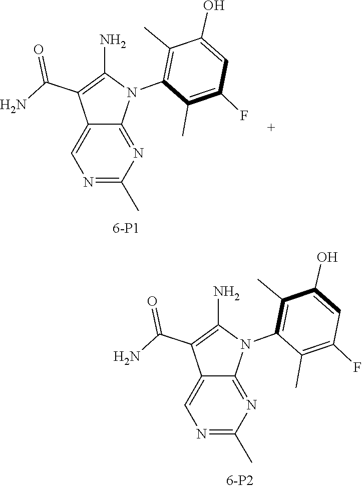

### Preparation of Intermediate 6-2′

To a solution of Intermediate 6-1′ (100 mg, 0.591 mmol) in NMP (2 mL) were added 5-bromo-4-chloro-2-methylpyrimidine (183.9 mg, 0.887 mmol) and methanesulfonic acid (168.7 mg, 1.755 mmol). The mixture was stirred at 90° C. under N₂ protected for 2 hr. The reaction mixture was cooled to room temperature, diluted with water (10 mL), extracted with EA (10 mL×3), and washed with brine (20 mL×3). The organic layer was dried over Na₂SO₄, filtered, and concentrated to dryness. The residue was purified by silica gel column chromatography to afford Intermediate 6-2′ (80 mg, 39.8%) as a yellow solid.

LCMS: 341.9 [M+H]⁺.

### Preparation of Intermediate 6-3′

To a solution of propanedinitrile (17.5 mg, 0.265 mmol) in DMF (2 mL) was added NaH (6.4 mg, 0.265 mmol) at 0° C. The reaction was stirred at room temperature for 30 min. Intermediate 6-2′ (90 mg, 0.265 mmol) and XantPhos Pd G3 (50.2 mg, 0.053 mmol) were added. The mixture was stirred at 130° C. for 2 hr. The reaction mixture was cooled to room temperature, diluted with water (10 mL), extracted with EA (15 mL×3), and washed with brine (20 mL×3). The organic layer was dried over Na₂SO₄, filtered, and concentrated to dryness. The residue was purified by silica gel column chromatography to afford Intermediate 6-3′.

LCMS: 326.2 [M+H]⁺.

### Preparation of Intermediate 6-4′

A solution of Intermediate 6-3′ (80 mg, 0.246 mmol) in H₂SO₄ (2 mL) was stirred at 25° C. under N₂ for 3 hr. The mixture was cooled to room temperature, carefully added into stirring ice-water (10 mL), and adjusted to pH˜8 with NaOH (4 M in water). The solution was extracted with DCM (15 mL×3). The combined organic layer was washed with brine (30 mL), dried over Na₂SO₄, filtered, and concentrated to dryness. The residue was purified by silica gel column chromatography to afford Intermediate 6-4′.

LCMS: 344.1 [M+H]⁺.

### Preparation of Compound 6

To a solution of Intermediate 6-4′ (77 mg, 0.225 mmol) in DCM (2 mL) was added tribromoborane (563.4 mg, 2.249 mmol) at 25° C., the mixture was stirred for 2 hr. The residue was purified by Pre-HPLC to afford racemic Compound 6. Chiral resolution of Compound 6 was conducted by Prep-SFC (Column: Daicel Chiralcel® OZ, 250*25 mm 10 μm; Condition: 22% MeOH (+0.1% 7.0 mol/L ammonia in MeOH) with 78% CO_{2;} Wavelength: 214 nm; Flow rate: 70 mL/min; Column temp: 25° C.; Back pressure: 100 bar). RT₁ (min): 3.42 (Compound 6-P1), RT₂ (min): 3.49 (Compound 6-P2).

LCMS: 330.1 [M+H]⁺;

¹H NMR (400 MHz, DMSO-d₆) δ 10.07 (s, 1H), 8.89 (s, 1H), 7.16 (s, 2H), 6.84 (d, J=3.2 Hz, 2H), 6.81 (s, 1H), 2.45 (s, 3H), 1.67 (s, 3H), 1.63 (s, 3H).

### Example 7

### Synthesis of Compound 7:

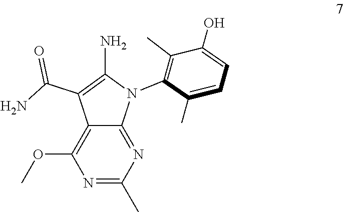

The title compound was synthesized according to the following synthetic procedures.

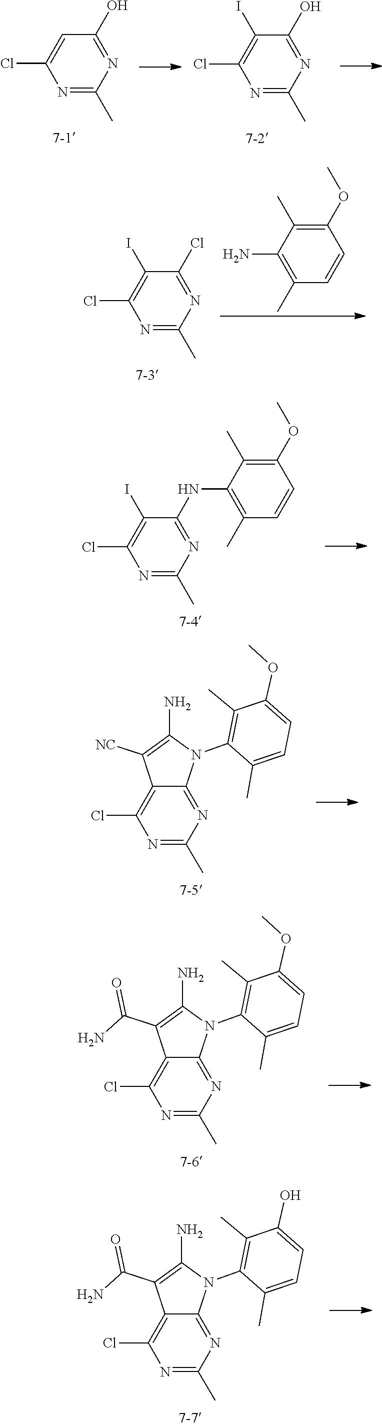

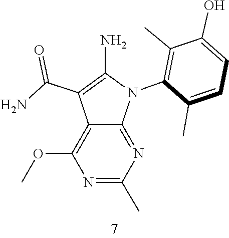

### Preparation of Intermediate 7-2′

To a solution of Intermediate 7-1′ (5 g, 34.587 mmol) in DMF (50 mL) was added NIS (15.6 g, 69.333 mmol). The mixture was stirred at 70° C. for 2 h. The mixture was diluted with water (50 mL) and sat.aq Na₂S₂O₃ (50 mL), extracted with EA (100 mL×3). Combined organic layers were washed with water (200 mL) and brine (200 mL), dried over Na₂SO₄, filtered and concentrated to dryness. The residue was purified by column chromatography on silica gel to afford Intermediate 7-2′ (8.7 g, 93.0%) as a yellow solid.

LCMS: 270.8 [M+H]⁺.

### Preparation of Intermediate 7-3′

To a solution of Intermediate 7-2′ (6.7 g, 24.769 mmol) in POCl₃ (70 mL) was stirred at 105° C. for 1 h. The reaction mixture was concentrated and diluted with EA (200 mL), which was washed with water (200 mL×3), dried over Na₂SO₄, filtered and concentrated to dryness. The residue was purified by silica gel column chromatography to afford Intermediate 7-3′ (6.2 g, 86.6%) as a white solid.

LCMS: 288.7 [M+H]⁺.

### Preparation of Intermediate 7-4′

To a solution of Intermediate 7-3′ (6.2 g, 21.461 mmol) and 3-methoxy-2,6-dimethylaniline (3.9 g, 25.794 mmol) in THF (100 mL) was added LiHMDS (42.9 mL, 42.922 mmol) at 0° C. The mixture was stirred at 0° C. for 1 h. The reaction mixture was carefully added into NH₄Cl (aq, 100 mL) under ice-bath, which was extracted with EA (100 mL×3). The combined organic layers were washed with brine (50 mL×3), dried over Na₂SO₄, filtered and concentrated to dryness. The residue was purified by silica gel column chromatography to afford Intermediate 7-4′ (2.15 g, 24.8%) as a yellow solid.

LCMS: 404.0 [M+H]⁺.

### Preparation of Intermediate 7-5′

To a solution of propanedinitrile (818.8 mg, 12.387 mmol) in DME (40 mL) was added NaH (495.5 mg, 12.388 mmol) at 0° C., the mixture was stirred at 0° C. for 30 min. Then to the mixture was added Intermediate 7-4′ (2.0 g, 4.955 mmol) and XantPhos Pd G₃ (470.4 mg, 0.495 mmol), the mixture was stirred at 130° C. for 4 h. The reaction mixture was diluted with water (500 mL), which was extracted with EA (500 mL×3). The combined organic layers were washed with brine (500 mL×3), dried over Na₂SO₄, filtered and concentrated to dryness. The residue was purified by silica gel column chromatography to afford Intermediate 7-5′ (700 mg, 41.3%) as a white solid.

LCMS: 342.2 [M+H]⁺.

### Preparation of Intermediate 7-6′

A mixture of Intermediate 7-5′ (100 mg, 0.325 mmol) in H₂SO₄ (4 mL) was stirred at 40° C. for 1 h. The mixture was poured into ice water (20 mL), the pH of which was adjusted to 8 with NaOH (4 M in water). The mixture was extracted with EA (30 mL×3). The combined organic layer was washed with brine (30 mL), dried over Na₂SO₄, filtered and concentrated to dryness. The residue was purified by silica gel column chromatography to afford Intermediate 7-6′.

LCMS: 360.2 [M+H]⁺.

### Preparation of Intermediate 7-7′

To a solution of Intermediate 7-6′ (155 mg, 0.431 mmol) in DCM (3 mL) was added BBr₃ (539.9 mg, 2.155 mmol). The mixture was stirred at 25° C. for 2 h. The residue was purified by reverse flash chromatography to afford compound Intermediate 7-7′.

LCMS: 346.1 [M+H]⁺.

### Preparation of Compound 7

To a solution of Intermediate 7-7′ (50 mg, 0.145 mmol) in MeOH (1 mL) was added NaOMe (23.5 mg, 0.435 mmol). The mixture was stirred at 50° C. under N₂ for 3 h. The reaction mixture was diluted with water (10 mL), which was extracted with EA (10 mL×3). The combined organic layer was washed with brine (20 mL), dried over Na₂SO₄, filtered and concentrated to dryness. The residue was purified by silica gel column chromatography and separated by prep-SFC and chiral SFC to afford Compound 7.

LCMS: 342.2 [M+H]⁺;

¹H NMR: (400 MHz, DMSO-d₆) δ 9.55 (s, 1H), 7.56 (s, 1H), 7.06 (d, J=8.2 Hz, 1H), 6.93 (s, 1H), 6.91 (s, 3H), 4.06 (s, 3H), 2.38 (s, 3H), 1.75 (s, 3H), 1.66 (s, 3H).

### Example 8

### Synthesis of Compound 8:

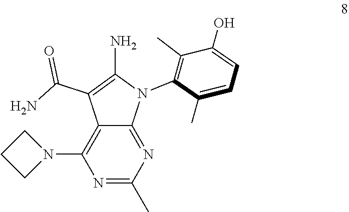

The title compound was synthesized according to the following synthetic procedures.

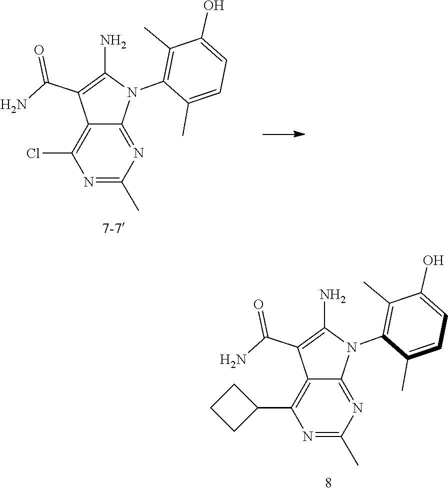

### Preparation of Compound 8

To a solution of Intermediate 7-7′ (50 mg, 0.145 mmol) in MeOH (1 mL) were added azetidine (16.6 mg, 0.290 mmol) and TEA (0.10 mL, 0.725 mmol). The mixture was stirred at 70° C. under N₂ for 3 h. The reaction mixture was diluted with water (10 mL), which was extracted with EA (10 mL×3). The combined organic layers were washed with brine (20 mL), dried over Na₂SO₄, filtered and concentrated. The residue was purified by silica gel column chromatography and further separated by prep-SFC and chiral SFC to afford Compound 8.

LCMS: 367.2 [M+H]⁺;

¹H NMR: (400 MHz, CD₃OD) δ 7.09 (d, J=8.3 Hz, 1H), 6.91 (d, J=8.3 Hz, 1H), 4.17 (t, J=7.5 Hz, 4H), 2.45 (s, 3H), 2.38-2.30 (m, 2H), 1.85 (s, 3H), 1.80 (s, 3H).

### Example 9

### Synthesis of Compound 9:

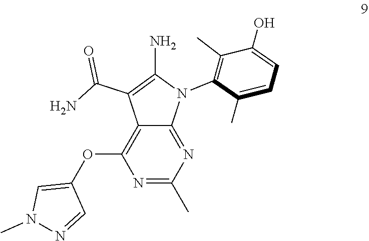

The title compound was synthesized according to the following synthetic procedures.

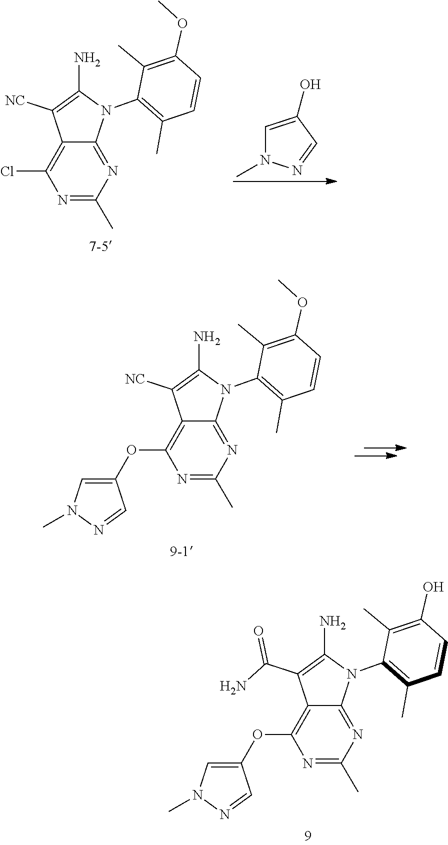

### Preparation of Intermediate 9-1′

A mixture of Intermediate 7-5′ (70 mg, 0.205 mmol) and 1-methyl-1H-pyrazol-4-ol (30 mg, 0.307 mmol), t-BuOK (69 mg, 0.614 mmol) was added in DMF (2 mL). The reaction was stirred at 120° C. for 16 h. The reaction mixture was diluted with water (10 mL), which was extracted with EA (10 mL×3). The combined organic layer was washed with brine (30 mL), dried over Na₂SO₄, filtered and concentrated to dryness. The residue was purified by silica gel column chromatography to afford Intermediate 9-1′.

LCMS: 404.2 [M+H]⁺.

### Preparation of Compound 9

In a similar fashion according to the procedure for Compound 1, Compound 9 was synthesized from Intermediate 9-1′.

LCMS: 407.9 [M+H]⁺;

¹H NMR: (400 MHz, DMSO-d₆) δ 9.56 (s, 1H), 7.96 (s, 1H), 7.54 (s, 1H), 7.39 (s, 1H), 7.08-6.96 (m, 4H), 6.93 (d, J=8.3 Hz, 1H), 3.85 (s, 3H), 2.33 (s, 3H), 1.77 (s, 3H), 1.68 (s, 3H).

### Example 10

### Synthesis of Compound 10:

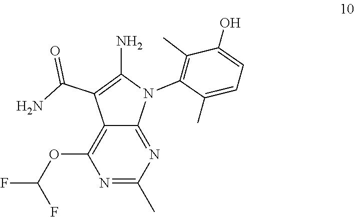 [SMILES: Cc1nc(OC(F)F)c2c(C(N)=O)c(N)n(-c3c(C)ccc(O)c3C)c2n1]

The title compound was synthesized according to the following synthetic procedures.

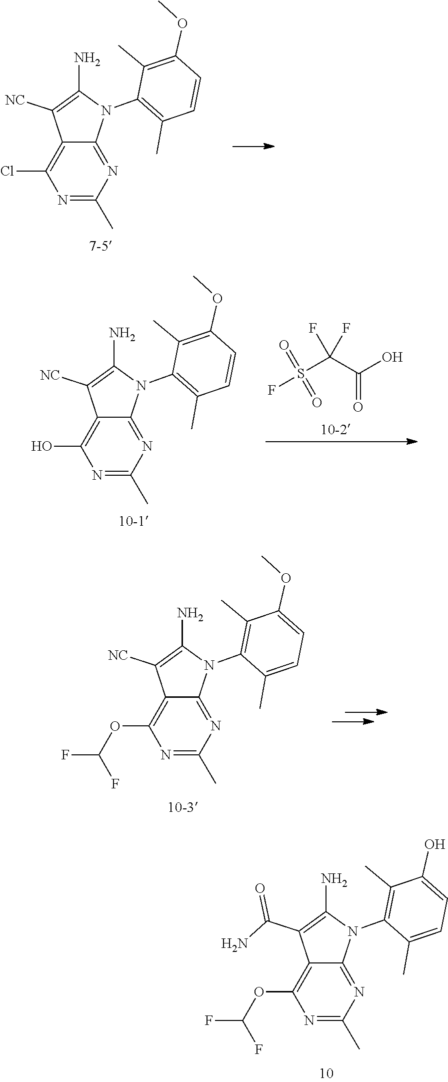

### Preparation of Intermediate 10-1′

To a solution of Intermediate 7-5′ (200 mg, 0.585 mmol) in AcOH (5 mL) was added NaOAc (192 mg, 2.341 mmol). The mixture was stirred at 100° C. for 16 h. The reaction mixture was carefully added into ice-water (30 mL) while stirring, which was adjusted to pH˜8 with NaOH (4M in water). The mixture was extracted with EA (30 mL×3). The combined organic layers were washed with brine (30 mL), dried over Na₂SO₄, filtered and concentrated to dryness. The residue was purified by silica gel column chromatography to afford Intermediate 10-1′ (125 mg, 66.1%) as a pink solid.

LCMS: 324.0 [M+H]⁺.

### Preparation of Intermediate 10-3′

To a solution of Intermediate 10-1′ (105 mg, 0.325 mmol) in ACN (4 mL) were added Intermediate 10-2′ (86.8 mg, 0.487 mmol) and Na₂CO₃ (68.9 mg, 0.650 mmol). The mixture was stirred at rt for 2 h. The mixture was extracted with EA (30 mL×3). The combined organic layers were washed with brine (30 mL), dried over Na₂SO₄, filtered and concentrated to dryness. The residue was purified by silica gel column chromatography to afford Intermediate 10-3′.

LCMS: 374.0 [M+H]⁺.

### Preparation of Compound 10

In a similar fashion according to the procedure for Compound 1, Compound 10 was synthesized from Intermediate 10-3′.

LCMS: 378.2[M+H]⁺.

¹H NMR: (400 MHz, DMSO-d₆) δ 9.70 (s, 1H), 8.40 (s, 1H), 7.90 (t, J=72.0 Hz, 1H), 7.21-6.92 (m, 6H), 2.41 (s, 3H), 1.75 (s, 3H), 1.67 (s, 3H).

### Example 11

### Synthesis of Compound 11:

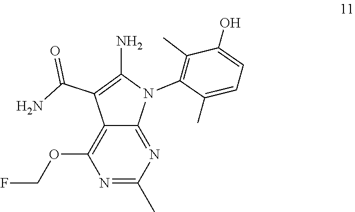 [SMILES: Cc1nc(OCF)c2c(C(N)=O)c(N)n(-c3c(C)ccc(O)c3C)c2n1]

The title compound was synthesized according to the following synthetic procedures.

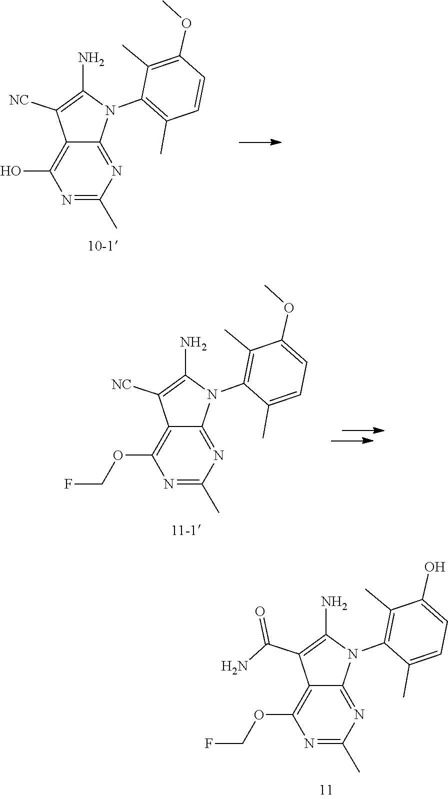 [SMILES: COc1ccc(C)c(-n2c(N)c(C#N)c3c(O)nc(C)nc32)c1C ; COc1ccc(C)c(-n2c(N)c(C#N)c3c(OCF)nc(C)nc32)c1C ; Cc1nc(OCF)c2c(C(N)=O)c(N)n(-c3c(C)ccc(O)c3C)c2n1]

### Preparation of Intermediate 11-1′

To a solution of Intermediate 10-1′ (150 mg, 0.464 mmol) in ACN (8 mL) were added fluoroiodomethane (89 mg, 0.556 mmol) and Cs₂CO₃ (181.4 mg, 0.557 mmol). The mixture was stirred at rt for 2 h. The mixture was filtered and concentrated to dryness. The residue was purified by silica gel column chromatography to afford Intermediate 11-1′ (60 mg, 36.4%).

LCMS: 356.2 [M+H]⁺.

¹H NMR: (400 MHz, DMSO-d₆) δ 7.25 (d, J=8.4 Hz, 1H), 7.13-7.10 (m, 3H), 6.23 (d, J=52.0 Hz, 2H), 3.85 (s, 3H), 2.40 (s, 3H), 1.80 (s, 3H), 1.71 (s, 3H).

### Preparation of Compound 11

In a similar fashion according to the procedure for Compound 1, Compound 11 was synthesized from Intermediate 11-1′.

LCMS: 360.2 [M+H]⁺.

¹H NMR: (400 MHz, CD₃OD) δ 7.11 (d, J=8.3 Hz, 1H), 6.93 (d, J=8.3 Hz, 1H), 6.31 (d, J=52.0 Hz, 2H), 2.48 (s, 3H), 1.86 (s, 3H), 1.80 (s, 3H).

### Example 12

### Synthesis of Compound 12, 12-P1, 12-P2:

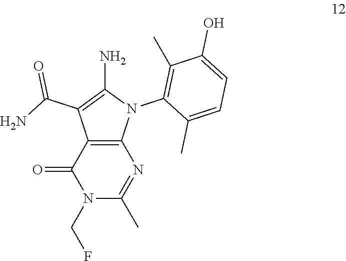 [SMILES: Cc1ccc(O)c(C)c1-n1c(N)c(C(N)=O)c2c(=O)n(CF)c(C)nc21]

The title compound was synthesized according to the following synthetic procedures.

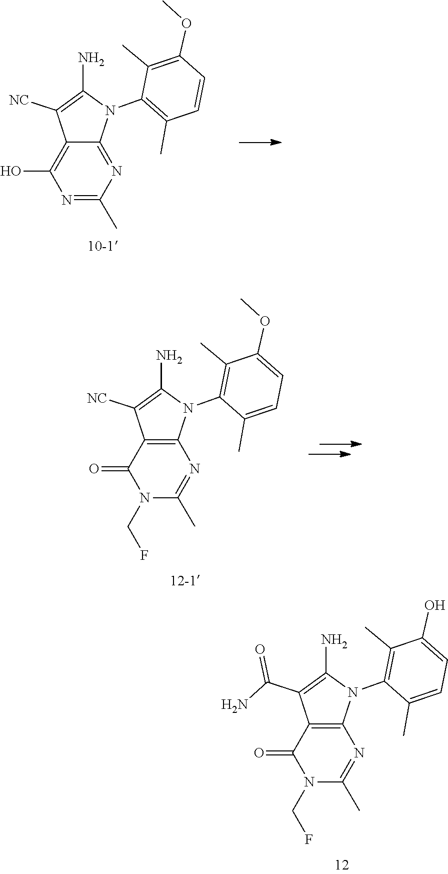 [SMILES: COc1ccc(C)c(-n2c(N)c(C#N)c3c(O)nc(C)nc32)c1C ; COc1ccc(C)c(-n2c(N)c(C#N)c3c(=O)n(CF)c(C)nc32)c1C ; Cc1ccc(O)c(C)c1-n1c(N)c(C(N)=O)c2c(=O)n(CF)c(C)nc21]

### Preparation of Intermediate 12-1′

Intermediate 12-1′ was another isomer separated in the preparation of Intermediate 11-1′ (85 mg, 51.5%).

LCMS: 356.2 [M+H]⁺.

¹H NMR: (400 MHz, DMSO-d₆) δ 7.23 (d, J=8.4 Hz, 1H), 7.09 (d, J=8.5 Hz, 1H), 6.59 (s, 2H), 6.18 (d, J=52.0 Hz, 2H), 3.84 (s, 3H), 2.45 (s, 3H), 1.84 (s, 3H), 1.74 (s, 3H).

### Preparation of Compound 12

In a similar fashion according to the procedure for Compound 1, Compound 12 was synthesized from Intermediate 12-1′.

LCMS: 360.0 [M+H]⁺;

¹H NMR: (400 MHz, DMSO-d₆) δ 9.58 (s, 1H), 9.14 (s, 1H), 7.06 (d, J=8.4 Hz, 1H), 6.92 (d, J=8.4 Hz, 1H), 6.88 (s, 1H), 6.54 (s, 2H), 6.23 (d, J=52.0 Hz, 2H), 2.49 (s, 3H), 1.80 (s, 3H), 1.71 (s, 3H).

The racemic compound 12 was separated by chiral-SFC to afford Compound 12-P1 (shorter retention time) and Compound 12-P2.

More compounds as listed below may be synthesized and may be also useful as PKMYT1 inhibitor.

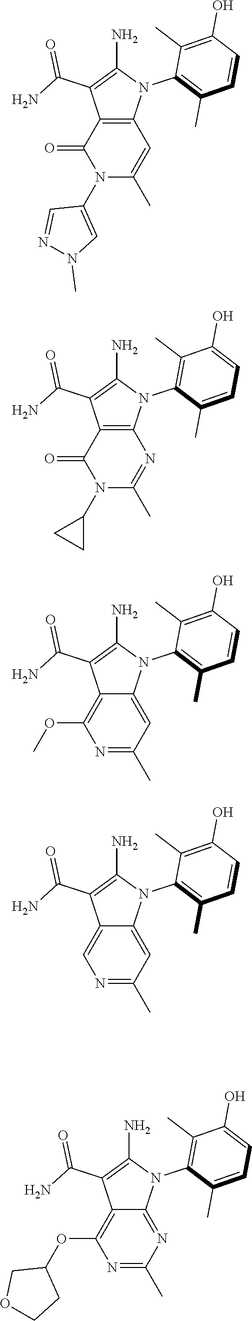

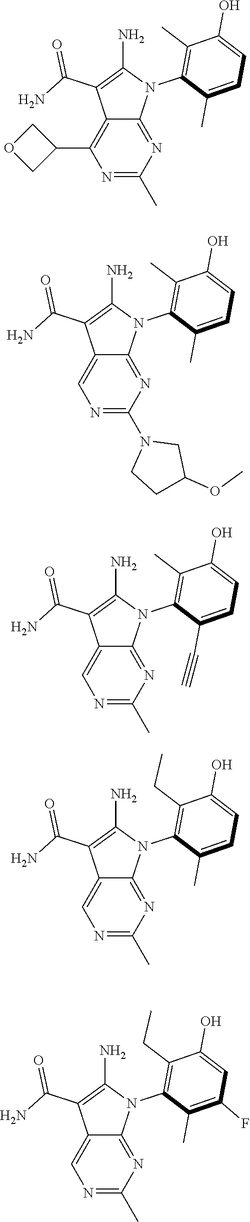

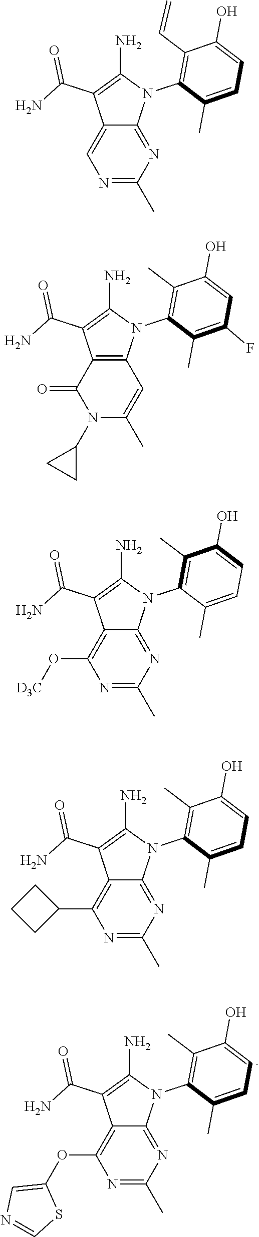

### Assay Example

### PKMYT1 HTRF Assay: Compounds IC₅₀ Determined Against PKMYT1

Compound serial dilution was performed by Echo, and the final concentrations varied from 10 μM to 0.5 nM. 5 μL/well of Enzyme solution was added to the assay plate containing the compound. The plate was centrifuged at 1000 rpm for 1 min, and incubated at 25° C. for 15 min. Then, 5 μL/well of tracer solution (Tracer 178) was added to initiate the reaction, and incubate for 60 min at 25° C. Next, 5 μL of GST-Tb was added into the assay plate, the plate was centrifuged at 1000 rpm for 1 min, and incubated at 25° C. for 15 min. The assay plate was read on Envision.

### WEE1 ADP-Glo Assay: Compounds IC₅₀ Determined Against WEE1

Compound serial dilution was performed by Echo, and the final concentrations varied from 10 μM to 0.5 nM. 5 μL/well of Enzyme solution was added to the assay plate containing the compound. The plate was centrifuged at 1000 rpm for 1 min, and incubated at 25° C. for 15 min. Then, 5 μL/well of substrate solution was added to initiate the reaction, and incubated for 60 min at 25° C. Next, 10 μL of kinase detection reagent was added into the assay plate, the plate was centrifuged at 1000 rpm for 1 min, and incubated at 25° C. for 60 min. The assay plate was read on Envision for US LUM as RLU.

The results from the PKMYT1 HTRF assay are exhibited in Table 2.

TABLE 2

| Compound ID | IC₅₀ (nM) |
|---|---|
| 1 | 3.0 |
| 1-P1 | 2.1 |
| 1-P2 | 46.1 |
| 2 | 8.9 |
| 3 | 11.9 |
| 4 | 4.1 |
| 5-P1 | 3.9 |
| 6-P1 | 1.8 |
| 6-P2 | 170.3 |
| 7 | 1.5 |
| 8 | 13 |
| 9 | 2.8 |
| 10 | 3.4 |
| 11 | 2.3 |
| 12-P2 | 1.5 |

The foregoing description is considered as illustrative only of the principles of the present disclosure. Further, since numerous modifications and changes will be readily apparent to those skilled in the art, it is not desired to limit the present disclosure to the exact construction and process shown as described above. Accordingly, all suitable modifications and equivalents may be considered to fall within the scope of the present disclosure as defined by the claims that follow.

All publications, patents and patent applications cited herein are incorporated by reference in their entirety into the disclosure.

1. A compound of Formula 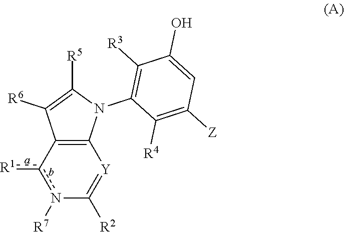
or a pharmaceutically acceptable salt, stereoisomer, or isotopic variant thereof,
wherein
“ [symbol] ” represents a single or double bond, provided that when “ [symbol] ” is a single bond, “ [symbol] ” is a double bond, or when “ [symbol] ” is a double bond, “ [symbol] ” is a single bond;
Y is N or CR⁸;
Z is hydrogen, optionally substituted C₁₋₃ alkyl, optionally substituted C₃₋₅ cycloalkyl, or halogen
when “ [symbol] ” is a single bond, R¹ is independently hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted 3- to 10-membered heterocyclyl, optionally substituted 3- to 10-membered heterocyclyl C₁₋₆ alkyl, optionally substituted 6- to 10-membered aryl, optionally substituted 5- to 10-membered heteroaryl, optionally substituted 5- to 10-membered heteroaryl C₁₋₆ alkyl, halogen, cyano, —N(R^{A})₂, —OR^{A}, —SR^{A}, —C(O)N(R^{B})₂, —SO₂N(R^{B})₂, —SO₂R^{C}, oxo (═O), or -Q-R^{X}; R⁷ is absent;
when “ [symbol] ” is a double bond, R¹ is independently O, S, or CH; R⁷ is hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted 3- to 10-membered heterocyclyl, optionally substituted 3- to 10-membered heterocyclyl C₁₋₆ alkyl, optionally substituted 6- to 10-membered aryl, optionally substituted 5- to 10-membered heteroaryl, optionally substituted 5- to 10-membered heteroaryl C₁₋₆ alkyl, halogen, cyano, —N(R^{A})₂, —OR^{A}, —C(O)N(R^{B})₂, —SO₂N(R^{B})₂, —SO₂R^{C}, or -Q-R^{X};
each of R², and R⁸ is independently hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted 3- to 10-membered heterocyclyl, optionally substituted 3- to 10-membered heterocyclyl C₁₋₆ alkyl, optionally substituted 6- to 10-membered aryl, optionally substituted 5- to 10-membered heteroaryl, optionally substituted 5- to 10-membered heteroaryl C₁₋₆ alkyl, halogen, cyano, —N(R^{A})₂, —OR^{A}, —SR^{A}, —C(O)N(R^{B})₂, —SO₂N(R^{B})₂, —SO₂R^{C}, oxo (═O), or -Q-R^{X};
each of R³ and R⁴ is independently optionally substituted C₁₋₆ alkyl, optionally substituted C₁₋₆ alkenyl, optionally substituted C₁₋₆ alkynyl, optionally substituted C₃₋₅ cycloalkyl, or halogen;
R⁵ is H or —N(R^{A})₂;
R⁶ is —C(O)NH(R^{B}), —C(O)R^{C}, or —SO₂R^{C};
each R^{X} is independently hydroxyl, optionally substituted C₁₋₆ alkyl, optionally substituted 6- to 10-membered aryl, optionally substituted 3- to 10-membered heterocyclyl, optionally substituted 5- to 10-membered heteroaryl, —N(R^{A})₂, —C(O)N(R^{B})₂, —SO₂N(R^{B})₂, —SO₂R^{C}, or optionally substituted C₁₋₆alkoxy;
each R^{A} is independently hydrogen, optionally substituted C₁₋₆ alkyl, optionally substituted 6- to 10-membered aryl C₁₋₆ alkyl, optionally substituted C₃₋₈ cycloalkyl, optionally substituted 6- to 10-membered aryl, optionally substituted 3- to 10-membered heterocyclyl, optionally substituted 5- to 10-membered heteroaryl, optionally substituted 5- to 10-membered heteroaryl C₁₋₆ alkyl, or —SO₂R^{C}; or two R^{A}, together with the atom to which they are attached, combine to form an optionally substituted 3- to 10-membered heterocyclyl;
each R^{B} is independently hydrogen, optionally substituted C₁₋₆ alkyl, optionally substituted C₂₋₆ alkoxy C₁₋₆ alkyl, optionally substituted 6- to 10-membered aryl C₁₋₆ alkyl, optionally substituted C₃₋₈ cycloalkyl, optionally substituted 6- to 10-membered aryl, optionally substituted 3- to 10-membered heterocyclyl, or optionally substituted 5- to 10-membered heteroaryl; or two R^{B}, together with the atom to which they are attached, combine to form an optionally substituted 3- to 10-membered heterocyclyl;
each R^{C} is independently optionally substituted C₁₋₆ alkyl, optionally substituted C₃₋₈ cycloalkyl, optionally substituted 6- to 10-membered aryl, optionally substituted 3- to 10-membered heterocyclyl, or optionally substituted 5- to 10-membered heteroaryl;
Q is optionally substituted C₁₋₆ alkylene, optionally substituted C₂₋₆ alkenylene, optionally substituted C₂₋₆ alkynylene, optionally substituted C₃₋₈ cycloalkylene, optionally substituted C₃₋₈ cycloalkenylene, optionally substituted 6- to 10-membered arylene, optionally substituted 3- to 10-membered heterocyclylene, or optionally substituted 5- to 10-membered heteroarylene.

2. The compound or a pharmaceutically acceptable salt, solvate, stereoisomer, or isotopic variant thereof of claim 1, wherein the compound is of Formula (I) or (II) 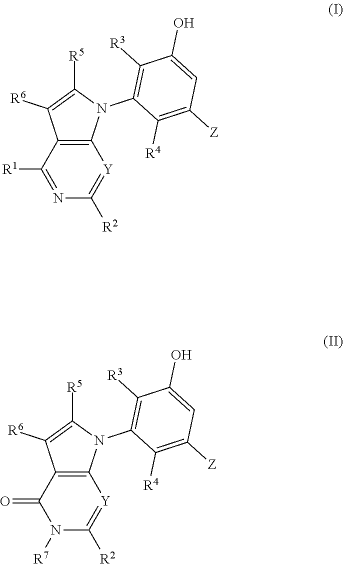
wherein
Y is N or CR⁸;
Z is hydrogen, optionally substituted C₁₋₃ alkyl, optionally substituted C₃₋₅ cycloalkyl, or halogen;
each of R¹, R², and R⁸ is independently hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted 3- to 10-membered heterocyclyl, optionally substituted 3- to 10-membered heterocyclyl C₁₋₆ alkyl, optionally substituted 6- to 10-membered aryl, optionally substituted 5- to 10-membered heteroaryl, optionally substituted 5- to 10-membered heteroaryl C₁₋₆ alkyl, halogen, cyano, —N(R^{A})₂, —OR^{A}, —SR^{A}, —C(O)N(R^{B})₂, —SO₂N(R^{B})₂, —SO₂R^{C}, or -Q-R^{X};
each of R³ and R⁴ is independently optionally substituted C₁₋₆ alkyl, optionally substituted C₁₋₆ alkenyl, optionally substituted C₁₋₆ alkynyl, optionally substituted C₃₋₅ cycloalkyl, or halogen;
R⁵ is H or —N(R^{A})₂;
R⁶ is —C(O)NH(R^{B}), —C(O)R^{C}, or —SO₂R^{C};
R⁷ is hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted 3- to 10-membered heterocyclyl, optionally substituted 3- to 10-membered heterocyclyl C₁₋₆ alkyl, optionally substituted 6- to 10-membered aryl, optionally substituted 5- to 10-membered heteroaryl, optionally substituted 5- to 10-membered heteroaryl C₁₋₆ alkyl, halogen, cyano, —N(R^{A})₂, —OR^{A}, —C(O)N(R^{B})₂, —SO₂N(R^{B})₂, —SO₂R^{C}, or -Q-R^{X};
each R^{X} is independently hydroxyl, optionally substituted C₁₋₆ alkyl, optionally substituted 6- to 10-membered aryl, optionally substituted 3- to 10-membered heterocyclyl, optionally substituted 5- to 10-membered heteroaryl, —N(R^{A})₂, —C(O)N(R^{B})₂, —SO₂N(R^{B})₂, —SO₂R^{C}, or optionally substituted C₁₋₆alkoxy;
each R^{A} is independently hydrogen, optionally substituted C₁₋₆ alkyl, optionally substituted 6- to 10-membered aryl C₁₋₆ alkyl, optionally substituted C₃₋₈ cycloalkyl, optionally substituted 6- to 10-membered aryl, optionally substituted 3- to 10-membered heterocyclyl, optionally substituted 5- to 10-membered heteroaryl, optionally substituted 5- to 10-membered heteroaryl C₁₋₆ alkyl, or —SO₂R^{C}; or two R^{A}, together with the atom to which they are attached, combine to form an optionally substituted 3- to 10-membered heterocyclyl;
each R^{B} is independently hydrogen, optionally substituted C₁₋₆ alkyl, optionally substituted C₂₋₆ alkoxy C₁₋₆ alkyl, optionally substituted 6- to 10-membered aryl C₁₋₆ alkyl, optionally substituted C₃₋₈ cycloalkyl, optionally substituted 6- to 10-membered aryl, optionally substituted 3- to 10-membered heterocyclyl, or optionally substituted 5- to 10-membered heteroaryl; or two R^{B}, together with the atom to which they are attached, combine to form an optionally substituted 3- to 10-membered heterocyclyl;
each R^{C} is independently optionally substituted C₁₋₆ alkyl, optionally substituted C₃₋₈ cycloalkyl, optionally substituted 6- to 10-membered aryl, optionally substituted 3- to 10-membered heterocyclyl, or optionally substituted 5- to 10-membered heteroaryl;
Q is optionally substituted C₁₋₆ alkylene, optionally substituted C₂₋₆ alkenylene, optionally substituted C₂₋₆ alkynylene, optionally substituted C₃₋₈ cycloalkylene, optionally substituted C₃₋₈ cycloalkenylene, optionally substituted 6- to 10-membered arylene, optionally substituted 3- to 10-membered heterocyclylene, or optionally substituted 5- to 10-membered heteroarylene.

3. The compound or a pharmaceutically acceptable salt, solvate, stereoisomer, or isotopic variant thereof of claim 2, wherein the compound is enriched for the atropisomer of Formula (IA) or (IIA):

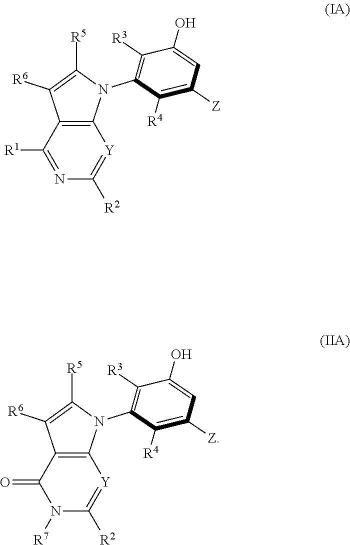

4. The compound or a pharmaceutically acceptable salt, solvate, stereoisomer, or isotopic variant thereof of claim 1, wherein the compound is of Formula (I′) or (II′) 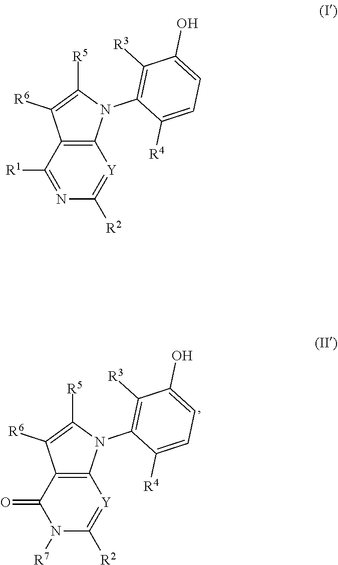
wherein
Y is N or CR⁸;
each of R¹, R², and R⁸ is independently hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted 3- to 10-membered heterocyclyl, optionally substituted 3- to 10-membered heterocyclyl C₁₋₆ alkyl, optionally substituted 6- to 10-membered aryl, optionally substituted 5- to 10-membered heteroaryl, optionally substituted 5- to 10-membered heteroaryl C₁₋₆ alkyl, halogen, cyano, —N(R^{A})₂, —OR^{A}, —SR^{A}, —C(O)N(R^{B})₂, —SO₂N(R^{B})₂, —SO₂R^{C}, or -Q-R^{X};
each of R³ and R⁴ is independently optionally substituted C₁₋₆ alkyl, optionally substituted C₁₋₆ alkenyl, optionally substituted C₁₋₆ alkynyl, optionally substituted C₃₋₅ cycloalkyl, or halogen;
R⁵ is H or —N(R^{A})₂;
R⁶ is —C(O)NH(R^{B}), —C(O)R^{C}, or —SO₂R^{C};
R⁷ is hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted 3- to 10-membered heterocyclyl, optionally substituted 3- to 10-membered heterocyclyl C₁₋₆ alkyl, optionally substituted 6- to 10-membered aryl, optionally substituted 5- to 10-membered heteroaryl, optionally substituted 5- to 10-membered heteroaryl C₁₋₆ alkyl, halogen, cyano, —N(R^{A})₂, —OR^{A}, —C(O)N(R^{B})₂, —SO₂N(R^{B})₂, —SO₂R^{C}, or -Q-R^{X};
each R^{X} is independently hydroxyl, optionally substituted C₁₋₆ alkyl, optionally substituted 6- to 10-membered aryl, optionally substituted 3- to 10-membered heterocyclyl, optionally substituted 5- to 10-membered heteroaryl, —N(R^{A})₂, —C(O)N(R^{B})₂, —SO₂N(R^{B})₂, —SO₂R^{C}, or optionally substituted C₁₋₆alkoxy;
each R^{A} is independently hydrogen, optionally substituted C₁₋₆ alkyl, optionally substituted 6- to 10-membered aryl C₁₋₆ alkyl, optionally substituted C₃₋₈ cycloalkyl, optionally substituted 6- to 10-membered aryl, optionally substituted 3- to 10-membered heterocyclyl, optionally substituted 5- to 10-membered heteroaryl, optionally substituted 5- to 10-membered heteroaryl C₁₋₆ alkyl, or —SO₂R^{C}; or two R^{A}, together with the atom to which they are attached, combine to form an optionally substituted 3- to 10-membered heterocyclyl;
each R^{B} is independently hydrogen, optionally substituted C₁₋₆ alkyl, optionally substituted C₂₋₆ alkoxy C₁₋₆ alkyl, optionally substituted 6- to 10-membered aryl C₁₋₆ alkyl, optionally substituted C₃₋₈ cycloalkyl, optionally substituted 6- to 10-membered aryl, optionally substituted 3- to 10-membered heterocyclyl, or optionally substituted 5- to 10-membered heteroaryl; or two R^{B}, together with the atom to which they are attached, combine to form an optionally substituted 3- to 10-membered heterocyclyl;
each R^{C} is independently optionally substituted C₁₋₆ alkyl, optionally substituted C₃₋₈ cycloalkyl, optionally substituted 6- to 10-membered aryl, optionally substituted 3- to 10-membered heterocyclyl, or optionally substituted 5- to 10-membered heteroaryl;
Q is optionally substituted C₁₋₆ alkylene, optionally substituted C₂₋₆ alkenylene, optionally substituted C₂₋₆ alkynylene, optionally substituted C₃₋₈ cycloalkylene, optionally substituted C₃₋₈ cycloalkenylene, optionally substituted 6- to 10-membered arylene, optionally substituted 3- to 10-membered heterocyclylene, or optionally substituted 5- to 10-membered heteroarylene.

5. The compound or a pharmaceutically acceptable salt, solvate, stereoisomer, or isotopic variant thereof of claim 4, wherein the compound is enriched for the atropisomer of Formula (IA′) or (IIA′):

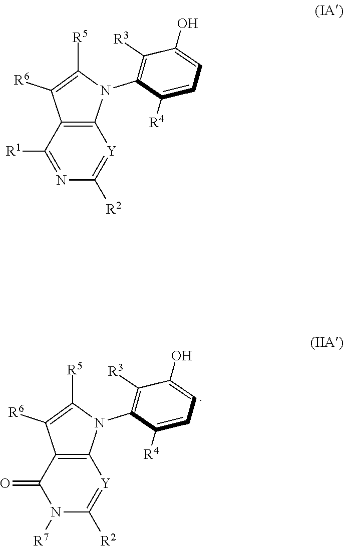

6. The compound or a pharmaceutically acceptable salt, solvate, stereoisomer, or isotopic variant thereof of claim 4, wherein Y is N.

7. The compound or a pharmaceutically acceptable salt, solvate, stereoisomer, or isotopic variant thereof of claim 4, wherein Y is CR⁸, and R⁸ is hydrogen or optionally substituted C₁₋₆ alkyl.

8. (canceled)

9. The compound or a pharmaceutically acceptable salt, solvate, stereoisomer, or isotopic variant thereof of claim 4, wherein R¹ is hydrogen, C₁₋₆ alkyl, C₃₋₈ cycloalkyl, 3- to 10-membered heterocyclyl, halogen, —OR^{A}′, or —SR^{A}′, in which R^{A}′ is C₁₋₆ alkyl, C₁₋₆ haloalkyl, C₃₋₈ cycloalkyl, optionally substituted 6- to 10-membered aryl, optionally substituted 3- to 10-membered heterocyclyl, or optionally substituted 5- to 10-membered heteroaryl.

10. The compound or a pharmaceutically acceptable salt, solvate, stereoisomer, or isotopic variant thereof of claim 4, wherein R¹ is hydrogen, fluoride, methoxy, monofluoromethoxy, difluoromethoxy, trifluoromethoxy, trifluoromethylmercapto, —OCD₃,

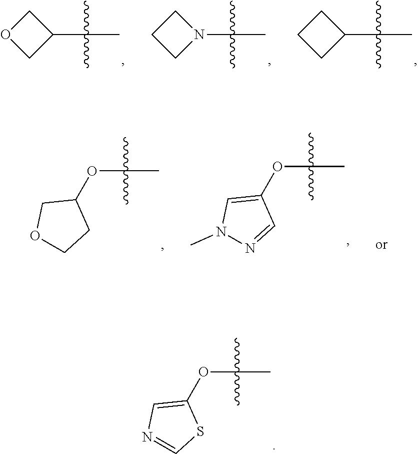

11. (canceled)

12. The compound or a pharmaceutically acceptable salt, solvate, stereoisomer, or isotopic variant thereof of claim 4, wherein R² is hydrogen, C₁₋₆ alkyl, C₃₋₈ cycloalkyl, or 3- to 10-membered heterocyclyl, in which said C₁₋₆ alkyl, C₃₋₈ cycloalkyl, and 3- to 10-membered heterocyclyl are optionally substituted by one, two, three, four, or five groups independently selected from the group consisting of halogen and C₁₋₆ alkoxy, as valency allows.

13. (canceled)

14. The compound or a pharmaceutically acceptable salt, solvate, stereoisomer, or isotopic variant thereof of claim 4, wherein R³ is C₁₋₆ alkyl, particularly methyl; or R³ is halogen, particularly chloride.

15. The compound or a pharmaceutically acceptable salt, solvate, stereoisomer, or isotopic variant thereof of claim 4, wherein R⁴ is C₁₋₆ alkyl, particularly methyl.

16. (canceled)

17. The compound or a pharmaceutically acceptable salt, solvate, stereoisomer, or isotopic variant thereof of claim 4, wherein R⁵ is —NH₂.

18. (canceled)

19. The compound or a pharmaceutically acceptable salt, solvate, stereoisomer, or isotopic variant thereof of claim 4, wherein R⁶ is —C(O)NH₂ or —C(O)NH(Me).

20. (canceled)

21. The compound or a pharmaceutically acceptable salt, solvate, stereoisomer, or isotopic variant thereof of claim 4, wherein R⁷ is hydrogen, optionally substituted C₁₋₆ alkyl, optionally substituted C₂₋₆ alkenyl, optionally substituted C₃₋₈ cycloalkyl, optionally substituted C₃₋₈ cycloalkenyl, optionally substituted 3- to 10-membered heterocyclyl, optionally substituted 6- to 10-membered aryl, or optionally substituted 5- to 10-membered heteroaryl.

22. (canceled)

23. The compound or a pharmaceutically acceptable salt, solvate, stereoisomer, or isotopic variant thereof of claim 4, wherein R⁷ is methyl, fluoromethyl, cyclopropyl, or 1-methyl-1H-pyrazol-4-yl.

24. (canceled)

25. The compound or a pharmaceutically acceptable salt, solvate, stereoisomer, or isotopic variant thereof of claim 1, wherein the compound is:

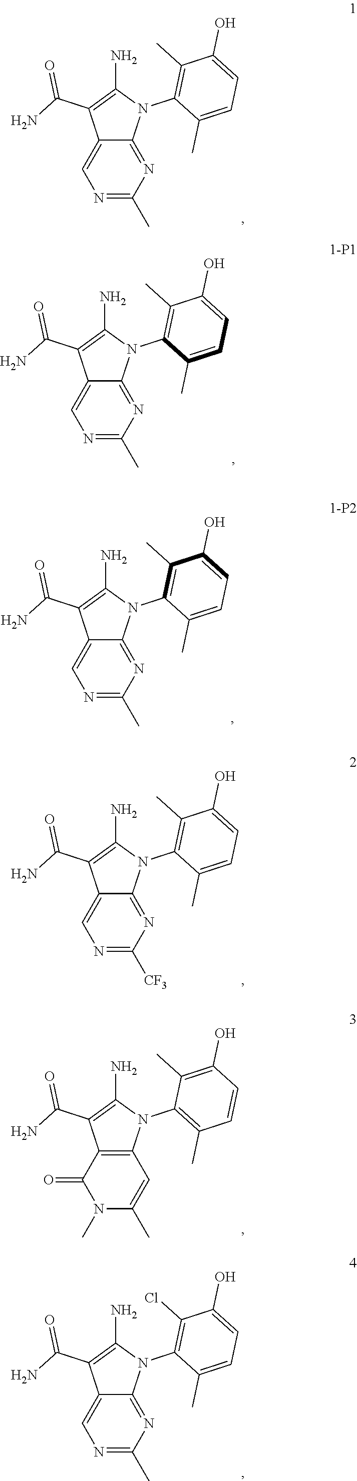

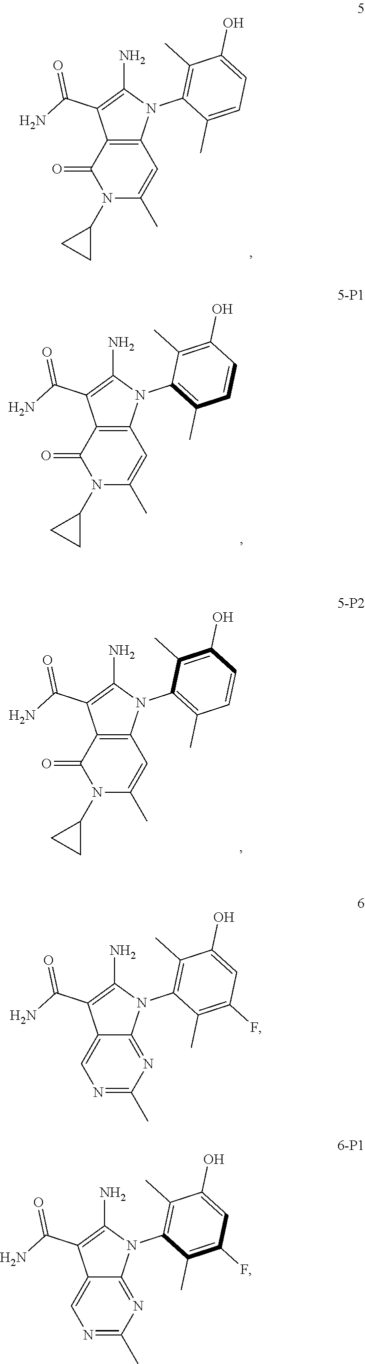

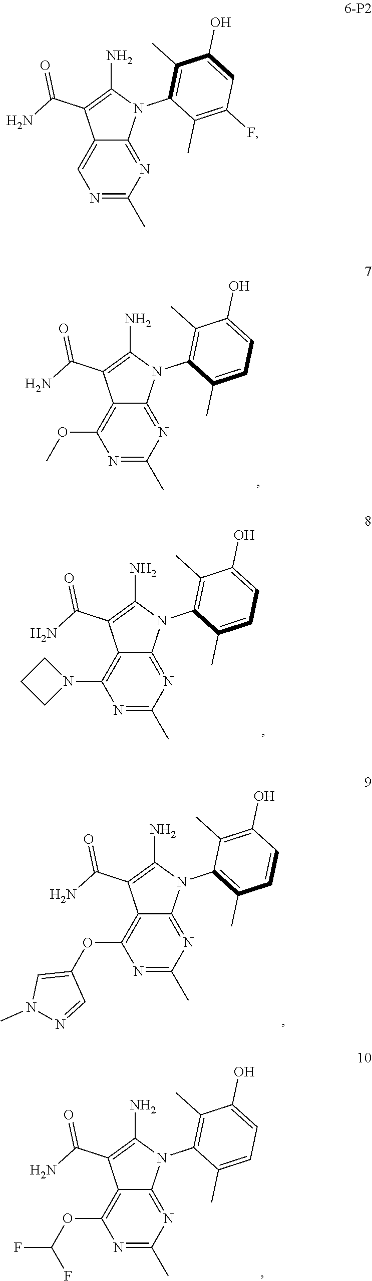

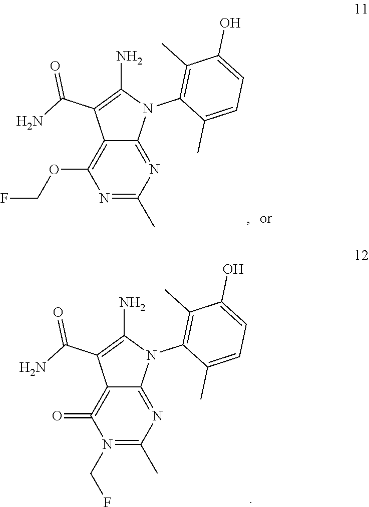

26. The compound or a pharmaceutically acceptable salt, solvate, stereoisomer, or isotopic variant thereof of claim 1, wherein the compound is:

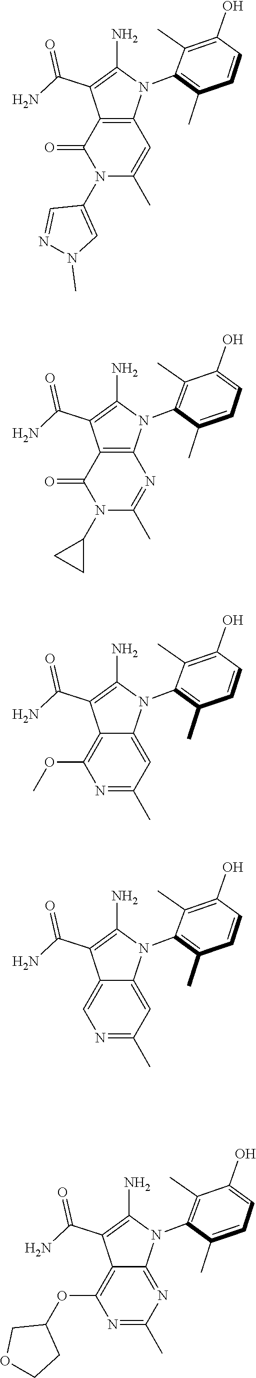

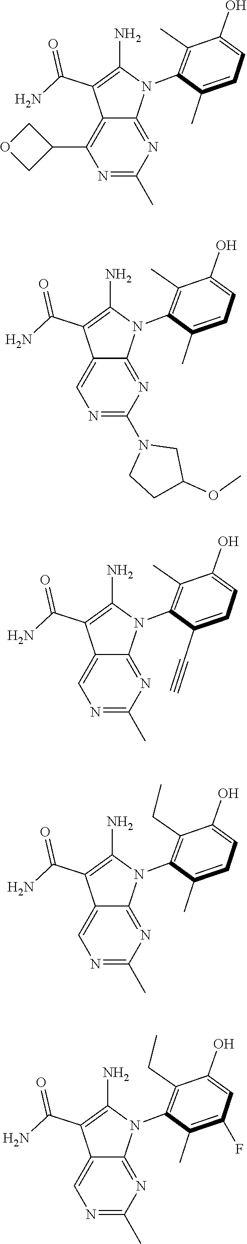

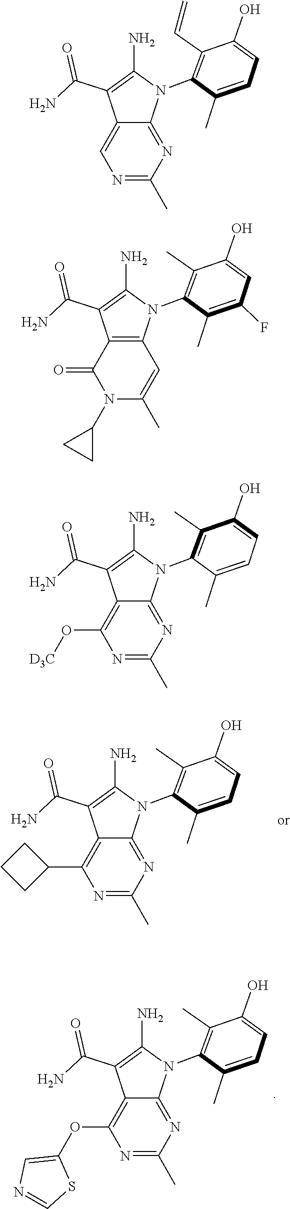

27. A pharmaceutical composition for treating a PKMYT1-associated disease or condition, which comprises the compound or a pharmaceutically acceptable salt, solvate, stereoisomer, or isotopic variant thereof of claim 1, and a pharmaceutically acceptable carrier or excipient.

28-29. (canceled)

30. A method of treating a PKMYT1-associated disease or condition in a subject in need thereof, which comprises administering to the subject a therapeutically effective amount of the compound or a pharmaceutically acceptable salt, solvate, stereoisomer, or isotopic variant thereof of claim 1.

31-41. (canceled)
